(19) United States
(12) Patent Application Publication
(10) Pub. No.: US 2016/0213824 A1
(43) Pub. Date: Jul. 28, 2016

(54) RECEPTACLE FOR BREAST MILK EXTRACTION

(71) Applicant: POSITIVE CARE LTD., Nazareth Ilit (IL)

(72) Inventor: Ofer FRIDMAN, Lotem (IL)

(21) Appl. No.: 14/915,261

(22) PCT Filed: Aug. 26, 2014
(86) PCT No.: PCT/IL14/50768

§ 371 Date: Feb. 29, 2016

Related U.S. Application Data
(63) Continuation of application No. 14/260,433, filed on Apr. 24, 2014.
(60) Provisional application No. 61/871,434, filed on Aug. 29, 2013.
(60) Provisional application No. 61/983,511, filed on Apr. 24, 2014.

(51) Int. Cl. A61M 1/06

(52) U.S. Cl. CPC A61M 1/064; A61M 1/066

(57) ABSTRACT

A receptacle for breast milk extraction using an external vacuum source and method of extraction are disclosed. The receptacle may include an open end for receiving a breast and a vacuum transfer zone on an outer surface thereof. The vacuum transfer zone may have a surface area of at least 1 cm² and the vacuum transfer area may be in pressure communication with an interior of the receptacle and the external vacuum source. A deformable diaphragm may block fluid communication between said interior of the receptacle and said external vacuum source. The diaphragm may limit a volume change associated pressure fluctuations. The receptacle and/or the diaphragm may be single use. Milk may optionally be collected into a flexible single use collection and/or storage vessel.

### RELATED APPLICATION/S

This application claims the benefit of priority under 35 USC §119(e) of U.S. Provisional Patent Application No. 61/871,434 filed 29 Aug. 2013, the contents of which are incorporated herein by reference in their entirety.

This application claims the benefit of priority under 35 USC §119(e) of U.S. Provisional Patent Application No. 61/983,511 filed 24 Apr. 2014, the contents of which are incorporated herein by reference in their entirety.

This application claims the benefit of priority from U.S. Utility patent application Ser. No. 14/260,433 filed 24 Apr. 2014.

This application is being co-filed with International Patent Application Agent Reference No. 59798.

The contents of all of the above applications are incorporated by reference as if fully set forth herein.

### FIELD AND BACKGROUND OF THE INVENTION

The present invention, in some embodiments thereof, relates to a breast milk extraction system and, more particularly, but not exclusively, to an extraction receptacle, diaphragm, a collection vessel and/or inter-connectors for the breast milk extraction system.

U.S. Pat. No. 5,358,476 discloses an infant nurser using a disposable, flexible plastic liner in a rigid, reusable shell. An adapter allows the breast milk to be pumped directly into the liner of the nurser without the need for any moving parts in response to cyclic pressure variations in the reservoir of the adapter. The adapter snaps onto the nipple-retaining cap and uses the liner of the nurser itself to form a valve. In a second embodiment, the adapter is provided with a unitary threaded circular rim to attach directly to the threaded end of the nurser shell. The adapter also includes a trigger for manual actuation of the valve when unmodulated negative pressure is applied to the reservoir.

U.S. Pat. No. 3,822,703 discloses a breast pump for use in stimulating lactation, particularly of the female human breast. The pump is equipped with a hollow chamber having a breast receiving opening adapted to engage at least the nipple area and means for pressurizing and depressurizing said chamber when in engagement with the breast. The chamber's breast receiving opening is fitted with a diaphragm having an aperture large enough for only the nipple area to extend through, said diaphragm being comprised of elastic material capable of vibrating back and forth in response to the pressurization and depressurization of the chamber when said diaphragm is in engagement with the breast.

### SUMMARY OF THE INVENTION

According to an aspect of some embodiments of the present invention there is provided a receptacle for breast milk extraction using an external vacuum source including: an open end for receiving a human breast; and a vacuum transfer zone on an outer surface of the receptacle, the vacuum transfer zone having a surface area of at least 1 cm² and the vacuum transfer zone being in pressure communication with an interior of the receptacle and the external vacuum source.

According to some embodiments of the invention, the receptacle further includes a deformable diaphragm blocking fluid communication between the interior of the receptacle and the external vacuum source wherein the deformable diaphragm is larger than the surface area and the deformable diaphragm is fixed across the vacuum transfer zone with a slack and wherein the slack is greater than an elasticity of the deformable membrane under a pressure differential of less than 250 mmHg across the membrane.

According to some embodiments of the invention, the receptacle further includes a deformable diaphragm blocking fluid communication between the interior of the receptacle and the external vacuum source wherein the deformable diaphragm is larger than the surface area and the deformable diaphragm is fixed across the vacuum transfer zone with a slack and wherein a movement of the deformable diaphragm is substantially limited to the slack.

According to some embodiments of the invention, the receptacle further includes a deformable diaphragm blocking fluid communication between the interior of the receptacle and the external vacuum source wherein the deformable diaphragm includes a portion of a collection vessel.

According to some embodiments of the invention, the receptacle further includes a deformable diaphragm blocking fluid communication between the interior of the receptacle and the external vacuum source wherein the deformable diaphragm is substantially non-elastic under pressure differences across the vacuum transfer surface of less than 250 mmHg.

According to some embodiments of the invention, the receptacle further includes a deformable diaphragm blocking fluid communication between the interior of the receptacle and the external vacuum source, and a containment for restricting movement of the deformable diaphragm to limit a volume shift between the receptacle and the external vacuum source associated with a pressure fluctuation transferred between the external vacuum source and the interior of the receptacle.

According to some embodiments of the invention, the receptacle further includes a deformable diaphragm blocking fluid communication between the interior of the receptacle and the external vacuum source, and a vacuum aperture along a pathway of the pressure communication and wherein the diaphragm obstructs the vacuum aperture when a volume shift between the receptacle and the external vacuum source associated with a pressure fluctuation surpasses a predetermined volume.

According to some embodiments of the invention, the receptacle further includes a connection configured for sealing to a milk collection vessel during the extraction such that the only opening between the milk collection vessel and an exterior of the receptacle is the open end.

According to some embodiments of the invention, a width of the open end is less than 50 mm.

According to some embodiments of the invention, the receptacle further includes a nipple chamber and wherein when a vacuum is applied to the vacuum transfer zone, a nipple of the breast is drawn into the nipple chamber and wherein a volume of the nipple chamber is less than 10 ml.

According to some embodiments of the invention, the length of the nipple chamber is less than 20 mm.

According to some embodiments of the invention, the receptacle further includes a diaphragm configured for: blocking fluid communication between the opening and the external vacuum source and limiting a volume shift between the receptacle and the external vacuum source associated with a pressure fluctuation transferred between the external vacuum source and the interior of the receptacle, wherein the diaphragm is configured for the limiting of the volume shift to less than a the volume of the nipple chamber.

According to some embodiments of the invention, the receptacle further includes a vacuum chamber rigidly connected to the outer surface of the receptacle and covering the vacuum transfer zone and wherein an inner volume of the vacuum chamber is in pressure communication with the vacuum transfer zone and the external vacuum source.

According to some embodiments of the invention, the vacuum chamber has an inner volume of at least 5 ml.

According to some embodiments of the invention, the receptacle further includes a nipple chamber and wherein when a vacuum is applied to the vacuum transfer zone, a nipple of the breast is drawn into the nipple chamber and wherein the vacuum chamber has an inner volume of at least 80% of a volume of the nipple chamber.

According to some embodiments of the invention, the receptacle further includes a deformable diaphragm blocking fluid communication between the interior of the receptacle and the external vacuum source and wherein the deformable diaphragm moves at least in part within the vacuum chamber.

According to some embodiments of the invention, the receptacle further includes a coupling reversibly restraining the vacuum chamber immobile with respect to the receptacle.

According to some embodiments of the invention, the receptacle further includes a fluid outlet; and a milk collection vessel receiving a fluid flow from the fluid outlet the milk collection vessel including a flexible portion covering a the vacuum transfer zone; the flexible portion blocking fluid communication between the interior of the receptacle and the vacuum chamber.

According to some embodiments of the invention, the flexible portion covers the fluid outlet allowing flow through the fluid outlet from the receptacle to the milk collection vessel and preventing flow through the fluid outlet from the milk collection vessel to the receptacle.

According to some embodiments of the invention, the vacuum chamber has a volume of at least 80% a volume of the receptacle.

According to an aspect of some embodiments of the present invention there is provided a method of extracting milk from a human breast using an external vacuum source including: mounting a receptacle to a breast, a portion of the breast received into an interior of the receptacle; shifting a volume between the receptacle and the external vacuum source to transmit a vacuum pressure fluctuation from external vacuum source to the interior of the receptacle and the breast thereby extracting the milk; blocking with a diaphragm fluid flow between the interior of the receptacle to the external vacuum source, and limiting the shifted volume to be less than a predetermined value by the diaphragm.

According to some embodiments of the invention, the limiting includes restricting movement of the diaphragm by means of a containment.

According to some embodiments of the invention, the diaphragm is substantially non-elastic in a vacuum range between 50 and 250 mmHg and the limiting includes restricting movement of the diaphragm to substantially to twice a volume of a slack in the diaphragm.

According to some embodiments of the invention, the shifting is via a diaphragm the limiting includes: obstructing a path of the vacuum signal by the diaphragm when the shifted volume reaches the predetermined value.

According to an aspect of some embodiments of the present invention there is provided a receptacle for extraction of milk from a human breast using an external vacuum source including: a breast opening in a first end for mounting over at least a portion of a human breast; a pressure communication pathway between the breast opening and the external vacuum source; a diaphragm configured for: blocking fluid communication between the opening and the external vacuum source and limiting a volume shift between the receptacle and said external vacuum source associated with a vacuum signal transferred along the pressure communication pathway.

According to some embodiments of the invention, the receptacle further includes a containment restricting a movement of the diaphragm.

According to some embodiments of the invention, the diaphragm is mounted across the pressure communication pathway with a slack and the slack is greater than a change in volume of the receptacle due to elasticity of the diaphragm over a pressure change of less than 250 mmHg.

According to some embodiments of the invention, the volume shift between the vacuum source and the receptacle is limited to substantially twice the slack.

According to some embodiments of the invention, the receptacle further includes a vacuum aperture on the pressure communication pathway and wherein the diaphragm obstructs the vacuum aperture when a volume of the vacuum signal exceeds a predetermined value.

According to some embodiments of the invention, the diaphragm includes a portion of a collection vessel.

According to some embodiments of the invention, the diaphragm is substantially non-elastic under pressure differences across the vacuum transfer surface of less than 250 mmHg.

According to some embodiments of the invention, the receptacle further includes a connection configured for sealing to a milk collection vessel during the extraction such that the only opening between the milk collection vessel and an exterior of the receptacle is the open end.

According to some embodiments of the invention, a width of the breast opening is less than 50 mm.

According to some embodiments of the invention, the receptacle further includes a nipple chamber and wherein when a vacuum is applied to the vacuum transfer zone, a nipple of the breast is drawn into the nipple chamber and wherein the length of the nipple chamber is less than 20 mm.

According to some embodiments of the invention, the receptacle further includes a nipple chamber and wherein when a vacuum is applied to the vacuum transfer zone, a nipple of the breast is drawn into the nipple chamber and wherein the a volume of the nipple chamber is less than 10 ml.

According to an aspect of some embodiments of the present invention there is provided a method of extracting milk from a human breast into a vessel including: mounting a breast opening at a first end of a milk extraction receptacle onto a breast; fitting a flexible portion of the vessel over an extension at second end of the milk extraction receptacle, the extension including a vacuum transfer zone; blocking fluid communication from inside the receptacle across the vacuum transfer zone to outside the receptacle with the flexible portion of the vessel; and communicating an oscillating vacuum signal from outside of the receptacle across the vacuum transfer zone into the receptacle by moving the flexible portion of the vessel.

According to some embodiments of the invention, the vessel is entirely flexible.

According to some embodiments of the invention, the method further includes forming a one way valve by draping the flexible portion of the vessel over a fluid outlet of the milk extraction receptacle.

According to some embodiments of the invention, the moving shifts a volume between the receptacle and the external vacuum source, the method further including: limiting a the shifted volume to be less than a predetermined value by restricting movement of the flexible portion of the vessel.

According to some embodiments of the invention, the restricting is by means of a containment.

According to some embodiments of the invention, the flexible portion of the vessel is substantially non-elastic in a vacuum range between 50 and 250 mmHg and the restricting is substantially to twice a volume of a slack in the flexible portion.

According to some embodiments of the invention, the method further includes obstructing a path of the vacuum signal by the flexible portion of the vessel when the shifted volume reaches the predetermined value.

According to an aspect of some embodiments of the present invention there is provided collection bag for a milk extraction receptacle including: a milk collection vessel; and a flexible conduit attachable to the extraction receptacle, the flexible conduit collapsing when a vacuum is applied from the extraction receptacle to prevent transmission of the vacuum to the collection vessel.

According to some embodiments of the invention, the flexible conduit has a wall thickness between 80 to 120 micron.

According to some embodiments of the invention, the flexible conduit is longer than the collection vessel.

According to an aspect of some embodiments of the present invention there is provided a method of collecting human breast milk including: mounting an open first end of a receptacle over the human breast; removably attaching a vacuum chamber rigidly to the receptacle over a vacuum transfer zone; blocking fluid flow between the receptacle and the vacuum chamber; and applying an alternating vacuum signal to the vacuum transfer zone to transfer the vacuum signal to the open first end of the receptacle to draw the milk from the breast to an outlet.

According to some embodiments of the invention, the blocking is by a diaphragm and the applying an alternating vacuum signal includes shifting a volume between the receptacle and the external vacuum source by moving the diaphragm the method further including: limiting a the shifted volume to be less than a predetermined value by restricting movement of the diaphragm.

According to some embodiments of the invention, the restricting is by means of a containment.

According to some embodiments of the invention, the diaphragm is substantially non-elastic in a vacuum range between 50 and 250 mmHg and the restricting is substantially to twice a volume of a slack in the diaphragm.

According to some embodiments of the invention, the blocking is by a diaphragm and the applying an alternating vacuum signal includes shifting a volume between the receptacle and the external vacuum source by moving the diaphragm the method further including: limiting a the shifted volume to be less than a predetermined value by obstructing a path of the vacuum signal with the diaphragm when the shifted volume reaches the predetermined value.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a flowchart illustrating a method of use of a milk extraction system in accordance with some embodiments of the current invention;

FIGS. 2A-C are flowcharts illustrating assembling a milk extraction system, operating the system and stowing the system after operation respectively in accordance with some embodiments of the current invention;

FIG. 3 is a chart of states of a milk extraction system in accordance with an embodiment of the present invention;

FIG. 4 is a block diagram illustrating components of a milk extraction system according to some embodiments of the current invention;

FIG. 5 is a schematic cross-sectional view of a milk extraction receptacle in accordance with some embodiments of the current invention;

FIGS. 6A and 6B are schematic cross sectional views of a breast milk collection system in negative pressure and positive pressure states respectively in accordance with an embodiments of the current invention;

FIGS. 7A and 7B are cutaway views of an alternative embodiment of a breast milk extraction system in accordance with some embodiments of the current invention;

FIG. 8 is a schematic view of a vacuum chamber attached to a collection receptacle in accordance with some embodiments of the present invention;

FIG. 9 is a perspective view of an extraction receptacle in accordance with some embodiments of the present invention;

FIG. 10 is a side view of a collection bag mounted onto an extraction receptacle in accordance with some embodiments of the present invention;

FIGS. 11A, 11B and 11C are perspective views of alternative embodiments of a frame for a diaphragm of a milk collection receptacle in accordance with some embodiments of the present invention;

FIGS. 11D, 11E and 11F are cutaway views of a device for limiting movement of a diaphragm by obstructing a pressure path in accordance with some embodiments of the present invention;

FIG. 12 is a side view of a collection receptacle connected to a rigid collection vessel in accordance with some embodiments of the current invention;

FIG. 13 is a schematic view showing dimensions of a flare and nipple chamber in accordance with some embodiments of the current invention;

FIG. 14 is a schematic view of an extraction receptacle in accordance with some embodiments of the current invention;

FIG. 15 is a schematic view of an extraction receptacle connected to a collection vessel and a vacuum source in accordance with some embodiments of the current invention; and

FIG. 16 is a schematic view of an adjustable volume extraction receptacle connected to a collection vessel and a vacuum source in accordance with some embodiments of the current invention.

### DESCRIPTION OF SPECIFIC EMBODIMENTS OF THE INVENTION

The present invention, in some embodiments thereof, relates to a breast milk extraction system and, more particularly, but not exclusively, to an extraction receptacle, diaphragm, a collection vessel and/or inter-connectors for the breast milk extraction system.

Overview

### 1) Protecting Milk from Previously Used Surfaces

Some aspects of the present invention relate to a breast milk extraction system protecting the breast milk from contaminated surfaces. Optionally, the breast milk is protected from contact with previously used surfaces. For example, the interface between extraction system and the breast may include a disposable extraction receptacle. For example, the milk may be extracted into a new and/or single use receptacle (for example a single use extraction receptacle) and/or collection vessel and/or storage vessel (for example a single use milk storage bag). Optionally, the single use receptacle and/or vessel will be separated from contact with reused components. For example, a single use extraction vessel may be separated from a reusable vacuum pump and/or vacuum chamber by a single use diaphragm. Optionally a single use parts may be made small and/or include inexpensive materials. For example the extraction receptacle, storage vessel and/or diaphragm may be made from cheap materials (for example thin Polyethylene which may be flexible and/or inelastic).

In some embodiments, a milk collection receptacle and/or a milk storage vessel may be made small and/or flexible. Optionally a large volume pressure reservoir may be contained by a vacuum chamber. For example, a pressure signal from a reusable vacuum chamber may be transmitted to a single use extraction receptacle by a diaphragm. In some embodiments, the volume of the vacuum chamber may range between 10 to 30% of the volume of the collection receptacle and/or 30 to 60% and/or 60 to 90% and/or 90 to 150% and/or more than 150% of the volume of the collection receptacle. For example the extraction receptacle and/or storage vessel may be made of polypropylene having a thickness ranging between 0.5 to 0.8 mm and/or 0.8 to 1.0 and/or 1.0 to 1.3 mm.

Optionally the pressure signal is transmitted over a large vacuum transmission zone. For example the vacuum transmission zone may have an area ranging between 1 cm² to 3 cm² and/or 3 cm² to 8 cm² and/or between 8 cm² to 15 cm² and/or more. The diaphragm may be made, for example, of thin, flexible and/or inelastic polyethylene (PE). For example the diaphragm may be made of the same material as a plastic milk storage bag. For example, the diaphragm may be a component of a milk storage bag. For example, the milk may be collected into a disposable collection vessel (for example a flexible plastic bag). For example, wall thickness of a collection vessel and/or the diaphragm may range for example less than 20 μm and/or between 20 to 50 μm and/or between 50 to 150 μm and/or between 150 to 300 μm and/or between 300 to 500 μm and/or greater than 500 μm. For example, for vessel having thickness less than 50 μm the material may be high density polyethylene. Alternatively or additionally, the collection receptacle and/or the storage vessel and/or the vacuum chamber may be made with metal and/or Polyethylene terephthalate (PET), Polyvinylidene chloride (PVDC), Polypropylene (PP), Polystyrene (PS), Polyamides (PA), Polycarbonate (PC) and/or other materials.

In some embodiments, an extraction receptacle may be small. For example the width (for example the diameter) of a breast opening of flare may range between 35 to 55 mm and/or between 55 to 65 mm and/or between 65 to 80 mm. In some embodiments, an extraction receptacle may have nipple channel (for example the nipple may sucked into and/or squeezed by the nipple channel during a negative pressure portion of a vacuum cycle). The length of the nipple channel may range for example between 5 to 15 mm and/or between 15 to 30 mm and/or between 30 to 50 mm and/or larger.

### 2) Extracting Breast Milk into a Flexible Collection and/or Storage Vessel

Some aspects of the present invention relate to a method of extracting breast milk into a flexible vessel. Optionally the milk extraction and/or storage vessel will be a plastic bag. Optionally, the bag will be from food grade bag materials. For example, a milk extraction receptacle may include a one way valve to allow milk to flow into a flexible vessel and/or prevent a vacuum from collapsing a flexible vessel. In some embodiments, a wall of the milk collection and/or storage vessel may include a diaphragm for an extraction receptacle. In some embodiments, a wall of the milk collection and/or storage vessel may include valve flap (for example for a one-way valve of an extraction receptacle). Alternatively or additionally, a collapsible conduit may carry milk from an extraction receptacle to a collection vessel. For example, milk may be pushed by positive pressure along the conduit. Optionally, in some embodiments milk is pushed upwards by positive pressure. Optionally, there may be an air inlet and/or an outlet. For example, the air inlet may be located near the beginning of a fluid path. For example a fluid outlet may be located towards the end of the collection system (for example near the collection vessel).

### 3) Diaphragm Limiting Volume of a Pressure Cycle

Some aspect of the present invention relate to a diaphragm for a vacuum pump. The diaphragm optionally transfers a pressure signal between an external vacuum source (for example a vacuum pump and/or a vacuum chamber) and a collection receptacle. For example the diaphragm may transfer a pressure signal between the vacuum source and the collection receptacle by shifting a volume between the source and the receptacle. The diaphragm optionally prevents cross contamination between the vacuum source and the collection receptacle. The diaphragm optionally limits the volume shifted between the collection receptacle and the vacuum source associated with transmission of a pressure signal between the vacuum source and the collection receptacle. For the sake of the current disclosure a pressure signal are changes of pressure over time. The changes may include increases of pressure and/or decreases in pressure. The pressure may include pressures greater than a gauge pressure and/or pressures less than a gauge pressure. For the sake of the present disclosure, a vacuum signal may include the portions of a pressure signal that are below gauge pressure. For the sake of the current disclosure, transmitting a pressure signal and/or a vacuum signal from a first space to a second space means decreasing pressure in the second space in response to at least some decreases in pressure of a pressure signal in the first space and/or increasing pressure in the second space is response to at least some increases in pressure of a pressure signal in the first space. For the sake of the current disclosure a volume shift associated with transmission of a pressure signal from a first space to a second space (which may be referred to as the “volume of the signal”) means the magnitude of the sum of the decrease in volume of the second space and/or volume of fluid shifted between the first and second space in response to a single pressure fluctuation in the first space.

In some embodiments, the volume associated with transmission of the pressure signal may be limited to a substantially fixed volume for a range of vacuum pressure fluctuation magnitudes. For example over a pressure and/or vacuum ranging between 30 to 120 mmHg and/or between 120 to 180 mmHg and/or between 180 to 250 mmHg the diaphragm may limit the volume associated with transmission of the signal to a volume ranging between 5 to 10 ml and/or between 10 and 20 ml and/or ranging between 20 to 40 ml and/or greater than 40 ml. For example, the diaphragm may limit the volume associated with transmission of a pressure signal to a volume that draws a nipple into a proper position in a nipple chamber. For example the volume shift associated with the vacuum signal may be limited to between 60% to 80% and/or 80% to 100% of the volume of a nipple chamber and/or between 100% to 150% and/or between 150% to 250% and/or greater than 250% the volume of a nipple chamber. For example the volume of the vacuum chamber may range between 60% to 80% and/or 80% to 100% of the volume of a nipple chamber and/or between 100% to 150% and/or between 150% to 250% and/or greater than 250% the volume of a nipple chamber. For example the volume of the vacuum chamber may range between 1 to 2 cm³ and/or 2 to 5 cm³ and/or 5 to 7 cm³ and/or 7 to 12 cm³ and/or 12 to 18 cm³ or more. Limiting the volume shift associated with transmission of a pressure signal may, for example, protect a nipple from being overextended and/or from being drawn beyond a designated space (for example a nipple chamber).

In some embodiments, the volume shift of a vacuum cycle transmitted by diaphragm may be limited by the elasticity and/or the slack on the diaphragm. For example, the volume of the vacuum signal may increase with the strength of the vacuum (for example by stretching diaphragm). Alternatively or additionally, the vacuum signal may be limited to a fixed volume. For example, diaphragm may have a fixed slack and/or a limited elasticity and/or when a certain volume has reached a vacuum aperture may be partially and/or completely obstructed, impeding and/or closing off a path of communication of the pressure signal. For example a containment may limit movement of diaphragm to a fixed and/or predetermined volume.

### 4) Coupling a Vacuum Chamber to an Extraction Receptacle

Some aspects of the present invention relate to coupling a vacuum source (for example a pump and/or a vacuum chamber) to a breast milk extraction receptacle. For example an extraction receptacle may include a mounting for a fastener. Optionally the coupling may be reversible and/or partially reversible (for example the coupling to a reusable part may be reversible and/or the coupling to a single use part may be irreversible. For example a fastener may include a quick connector. Optionally, a vacuum chamber is held rigidly to a vacuum transfer zone of the receptacle. For example a fastener may include a strap and/or a clip and/or a cone fitting and/or a b-spring clip and/or a slotted spring pin (e.g. a c-pin) and/or other fasteners.

### 5) Range of Possible Volumes for the Vacuum Chamber

In some embodiments, a receptacle opening in a vacuum chamber may be in pressure communication with a vacuum transfer zone in an extraction receptacle. For example, one or both of the openings of the vacuum chamber and/or pressure transfer zone of the extraction receptacle may include one and/or many holes. The vacuum transfer zone and/or the opening of the pressure chamber and/or each individual hole may have any shape (for example rectangular and/or having rounded corners and/or oval. For example, one or both of the openings of the vacuum chamber and/or pressure transfer zone of the extraction receptacle may have an area ranging, for example, between 1 cm² to 3 cm² and/or 3 cm² to 8 cm2 and/or between 8 cm² to 15 cm² or more. For example, the vacuum chamber opening may fit vacuum transfer zone of the extraction receptacle. Optionally, the opening of the vacuum chamber and/or pressure transfer zone of the extraction receptacle will be partially and/or completely surrounded by a sealing surface. Optionally, the sealing surface will be configured to facilitate produce an air tight connection between the extraction receptacle and a vacuum chamber. In some embodiments, a diaphragm may prevent mass transport between vacuum chamber and extraction receptacle. Optionally, diaphragm may pass a pressure signal between the pressure source and the extraction receptacle.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways. Similar steps and/or components and/or aspects may be labeled with the same numbering in multiple figures. Descriptions of options, components, steps and/or aspects with regard to one figure apply also to similarly labeled options, components, steps and/or aspects of other figures.

### Exemplary Embodiments

### 1) Method of Extracting Breast Milk

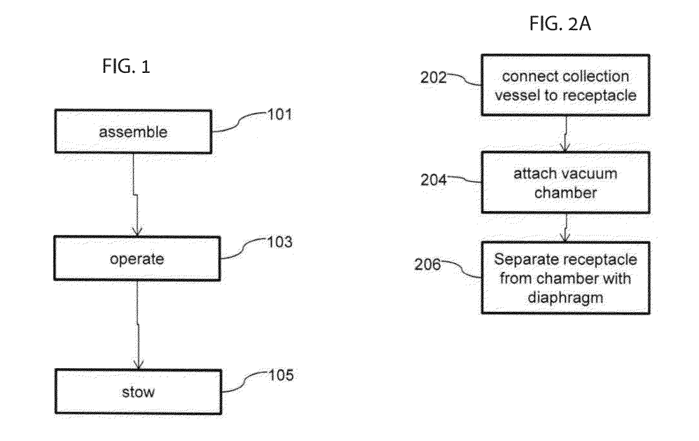

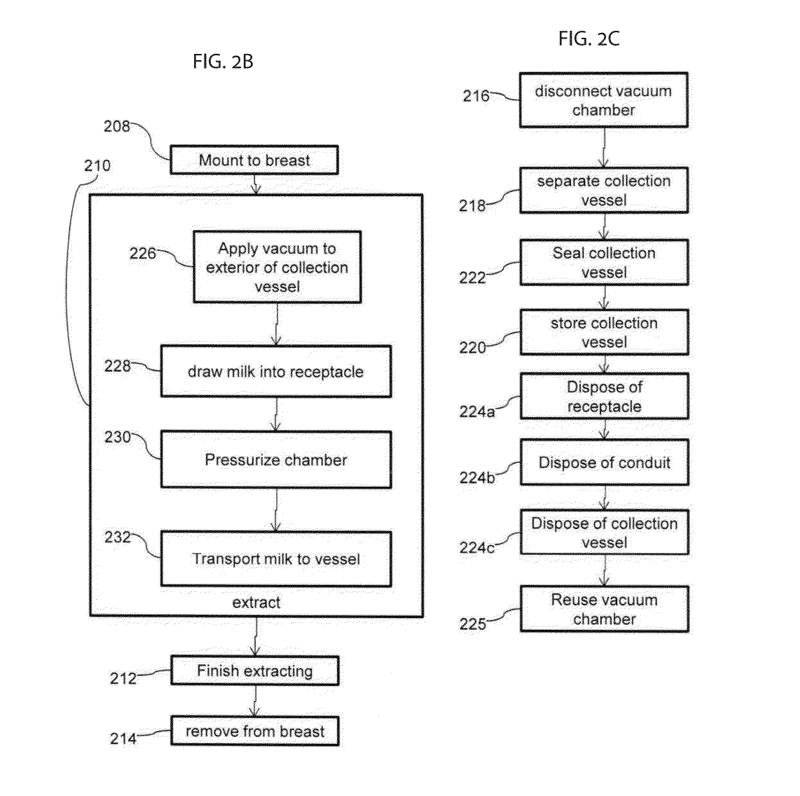

Referring now to the drawings, FIG. 1 illustrates a method of assembling 101 a milk extraction system, operating 103 the system and stowing 105 the system after operation in accordance with some embodiments of the current invention. Optionally (for example as illustrated in FIG. 2), a vacuum chamber is rigidly attached 204 to a milk extraction receptacle. A diaphragm optionally separates 206 the vacuum chamber from the extraction receptacle. In some embodiments, the diaphragm may be in place already when the vacuum chamber is attached 204 to the flare. In some embodiments the diaphragm may include a portion of a collection vessel. For example, the collection vessel may be a flexible bag and/or the diaphragm may include a portion of the wall of the bag. Optionally components that are exposed to milk (for example the collection vessel and/or the extraction receptacle and/or the diaphragm) are disposable and/or single use. Optionally components that are not disposable and/or single use (for example the vacuum chamber) do not contact the milk. Optionally, some or all of the disposable components may be permanently attached together. Optionally reusable components may be detachably connected to the disposable components. Detachment may optionally be reversible and/or irreversible.

### 2) Extracting and Collecting

FIG. 2A illustrates a method of assembling 101 a milk extraction system prior to use according to some embodiments of the current invention. For example, the user may connect 202 the collection vessel to the extraction receptacle. For example the collection vessel may include a flexible bag. A part of the bag may fit the collection vessel. The bag may be connected to the receptacle, for example by a friction fitting and/or by an adhesive and/or by an elastic coupler and/or a portion of the collection vessel may be sandwiched between the extraction receptacle and another element (for example a pressure chamber). In some embodiments, some components of an extraction system may be supplied preassembled. For example, a collection vessel may be preattached to a collection receptacle. For example the bag may be connected to the receptacle by an irreversible means, the bag and receptacle may be molded together and/or connected by an adhesive.

In some embodiments, a vacuum chamber may be attached 204 to the extraction receptacle. Attachment 204 of the vacuum chamber to the extraction receptacle may be rigid. For example between 50% and 100% of the volume of the vacuum chamber may be immobile with respect to the extraction receptacle. Attachment between the vacuum chamber and the extraction receptacle may be for example by a friction fitting and/or by an adhesive. Optionally, when the vacuum chamber and/or the collection receptacle are attached 204 together, openings in the receptacle and chamber may be aligned. Pressure signals may be transmitted across the vacuum transfer zone between the pressure chamber and the collection receptacle.

In some embodiments, a diaphragm may separate 206 between the vacuum chamber and the extraction receptacle. For example, the diaphragm may cover the hole between the extraction receptacle and the vacuum chamber. Optionally, the diaphragm may protect the vacuum chamber from contact with milk passing through the collection receptacle and/or the diaphragm may protect the milk in the collection receptacle from contact with the vacuum chamber. For example a portion of the collection vessel may be sandwiched between the collection receptacle and the vacuum chamber. Optionally friction between the collection receptacle, the collection vessel and/or the vacuum chamber may hold some and/or all of the components in place.

FIG. 2B illustrates a method of operating 103 a milk extraction system according to some embodiments of the current invention. In some embodiments, an extraction receptacle may be mounted 208 to a breast and/or milk may be extracted 210 from the breast. Alternatively or additionally, the collection receptacle may remain on the breast and the collection system may be assembled while attached to the breast Milk collection may include, for example alternatively applying a vacuum and/or a neutral pressure and/or a positive to the breast and/or a nipple and/or an areola. Alternatively or additionally, collecting milk may include drawing the nipple into a tube and/or a small passageway. Alternatively or additionally, collecting may include squeezing and/or massaging of the nipple, the areola and/or other parts of the breast. Alternatively or additionally, collecting may include transferring the milk to the collection vessel (for example by gravity flow and/or by positive pressure flow).

In some embodiments, the milk is protected from contact with previously used surfaces. The sides of the collection vessel optionally serve as diaphragm separating between a fluid flow region and a reusable component (for example a vacuum chamber and/or a pump). A portion of the bag optionally serves as a one way valve and/or as a valve flap (for example directing vacuum to the breast and away from a flexible collection vessel) and/or a portion of the bag optionally serves as a conduit (for example for transporting the milk from the extraction receptacle to collection vessel). Transport of the milk from the receptacle to the storage container may optionally be by positive pressure.

In some embodiments, milk may be extracted from a breast by applying 226 a vacuum to an external surface of a collection vessel. For example, the collection vessel may include a flexible portion. Optionally, the flexible portion may form part of a diaphragm covering the vacuum transfer zone and/or an opening between a vacuum chamber and an extraction receptacle. For example, a portion of the bag may be shaped to fit in and/or around a portion of the receptacle. Optionally the receptacle may include a rigid air pressure pathway between the breast flare and an opening to the vacuum chamber. When a vacuum is applied to the vacuum chamber the diaphragm optionally moves in the space of the opening between the vacuum chamber and the breast flare, transferring the pressure changes from the vacuum chamber to the breast flare. Additionally or alternatively a one way valve may prevent escape of the vacuum to the collection vessel. For example, a portion of the collection vessel may form a flap of a one-way valve that allows milk to flow from the extraction receptacle to the extraction vessel and/or prevents flow from the vessel to the receptacle. Alternatively or additionally, the receptacle and/or the vacuum chamber may include a fastener for attaching the vacuum chamber to the receptacle. Alternatively or additionally, the receptacle and/or the vacuum chamber may be designed to limit movement of the diaphragm, limiting the volume change of the breast flare (for example, protecting the nipple from being damaged and/or sucked too deeply into the receptacle).

In some embodiments, a vacuum applied 226 to the exterior of the collection vessel and/or extraction receptacle is transferred to the interior of the extraction receptacle and/or to the breast. For example the pressure signal may draw 228 milk into the receptacle (for example the vacuum may directly suck milk from the breast and/or milk may be squeezed out of a nipple [for example by drawing the nipple into a nipple channel]). The vacuum may close a one way valve preventing leak of a pressure from the receptacle. Optionally, the vacuum may be applied in cycles over a time period ranging, for example between 30 sec to 60 sec and/or 60 sec to 120 sec. and/or more than 120 sec. Optionally the vacuum cycles may be applied in a manner that increases over time until a target magnitude is reached. Optionally, during a significant portion of an extraction session the target magnitude of the vacuum ranges for example between 50 to 90 mmHg and/or from 90 to 130 mmHg and/or between 130 and 180 mmHg and/or between 180 and 250 mmHg. For example, the significant portion of the extraction session may be range between 3 to 5 minutes and/or 5 to 10 minutes and/or 10 to 15 minutes and/or 15 to 20 minutes and or 20-40% of the extraction session and/or 40 to 70% of the extraction session and/or more than 70% of the extraction session. For example in the beginning of the session the negative pressure portion of the cycle may reach a lower magnitude of vacuum. Later in the extraction session, the negative pressure portion of the cycle may reach a higher magnitude of vacuum. In some embodiments vacuum extraction may be coupled with squeezing and/or massaging. While the breast is squeezed, a lower vacuum magnitude may be used than when there is no squeezing and/or massaging.

In some embodiments, the pressure cycle may have a positive portion. For example, the vacuum chamber may be pressurized 230. Pressure in the vacuum chamber may push the diaphragm (for example a portion of the wall of the collection vessel) into the extraction receptacle, reducing the volume of the receptacle and/or increasing the pressure in the receptacle. During the positive portion of the cycle milk is optionally pushed through a one-way valve towards a milk collection portion of the milk collection vessel. For example, milk may be transported 232 to the collection area in a collapsible conduit. Positive pressure may help insure that the conduit stays open and/or opens just when enough positive pressure is applied. For example the conduit may stay open at a small vacuum (for example of less than 30 mmHg) and/or at a low positive pressure (for example from 0 to 10 mmHg) and/or at positive pressure of between 10 and 50 mmHg and/or at a positive pressure of greater than 50 mmHg. In some embodiments, a conduit from the extraction receptacle to the collection area may have uphill portions. Positive pressure may help push the milk up the uphill portion of the conduit. Optionally a retainer may hold the extraction receptacle to the breast (for example preventing the receptacle from being displaced during the positive portion of the pressure cycle). In some embodiments the cycle length may range for example between of 0.5 to 1 sec and/or 1 to 3 sec and/or 3 to 6 sec or more. The positive pressure may range for example between 0.1 to 30 mmHg and/or between 30 to 60 mmHg and/or between 60 to 250 mmHg and the vacuum may range for example between 0.1 to 30 mmHg and/or between 30 to 60 mmHg and/or between 60 to 120 and/or between 120 to 180 and/or between 180 to 250 mmHg.

In some embodiments, collection may be finished 212 and/or some extraction may be stopped and/or part of the collection system may be removed 214 from the breast and/or stowed 105 for example including disassembly. Optionally, an extraction session may be finished 212 and/or stopped after a predetermined period of time (for example ranging between 5 minutes to 15 minutes and/or 15 minutes to a half hour), and/or when the rate of milk production is small (for example when the rate of milk production is reduced below a value ranging between 1 ml/min to 5 ml/min and/or between 5 ml/min to 10 ml/min) and/or when the rate drops below a certain percentage of the initial extraction rate (for example between 2% to 5% of the initial rate and/or between 5% to 15% and/or between 15% to 30% and/or between 30% to 50% of the initial rate). Alternatively or additionally milk extraction may be finished when the external volume of the breast is reduced by predetermined amount and/or when the breast becomes soft and/or when there is a combination of factors. Alternatively or additionally, an extraction session may end and/or the flare may be removed and/or a collection vessel may be changed after predetermined extracted volume.

In some embodiments, the receptacle, collection vessel and/or vacuum chamber are all removed 214 from the breast together. Alternatively or additionally, some parts of the collection assembly may remain in contact with the breast while other components are disassembled and/or removed 214 from the breast. For example the extraction receptacle may remain on the breast while the collection vessel is removed 214 and/or the vacuum chamber is disconnected 216 (for example as illustrated in FIG. 2C). In some embodiments removal of some components may be reversible and/or irreversible. For example, a reusable collection vessel may be reversibly attached 204 and/or detached 218 from the extraction the receptacle by a screw thread. Optionally, the vacuum chamber and/or the collection vessel may be attached 204 and/or disconnected 216 using a clamp and/or a friction fitting. Alternatively or additionally, disattachment 218 of some components may be irreversible. For example, a collection vessel may be detached 218 from the extraction receptacle by tearing a connecting conduit.

In some embodiments, the collection vessel and/or milk may be stored 220.

Optionally the collection vessel is sealed 222 and/or labeled for storage. For example, a collection vessel may be sealed 222 by removable cap (for example a screw on cap) and/or a reversible seal (for example a plastic zipper and/or a twist tie and/or an adhesive) and/or irreversibly (for example by tying off an exit conduit and/or by heat sealing). For example the extraction system may include a heat sealer.

In some embodiments, some components of the extraction system may be disposed of 224 after a single and/or multiple uses. For example the collection receptacle and/or the diaphragm may be disposed of 224 after a single use. Alternatively or additionally the collection vessel may be used to store milk and later disposed. For example, when a collection vessel is removed 214 from the extraction receptacle, the fastener may be permanently disabled. When extraction is finished 212 the receptacle is optionally disposed of 224a. Alternatively or additionally, a conduit and/or sleeve connecting the collection vessel to the extraction receptacle may be single use and/or may be disposed of 224b after user. The collection vessel is then optionally used for storing the extracted milk. Optionally the collection vessel is single use and is disposed of 224c after the milk is consumed. Alternatively or additionally, the receptacle and/or the conduit and/or the sleeve and/or the collection vessel and/or a storage vessel may be reused. In some embodiments, the vacuum chamber may be reused 225.

FIG. 2C illustrates a method of stowing 105 a milk extraction system after use according to some embodiments of the current invention. In some embodiments, collection may be finished 212 and/or some extraction may be stopped and/or part of the collection system may be removed 214 from the breast and/or disassembled. In some embodiments, the receptacle, collection vessel and/or vacuum chamber are all removed 214 from the breast together. Alternatively or additionally, some components of the collection assembly may remain in contact with the breast while other components are disassembled and/or removed 214 from the breast. For example, a collection vessel may be irreversibly removed 214 from the extraction receptacle by tearing a connecting section. Alternatively or additionally the receptacle and collection vessel may be stored and/or disposed of 224 together

In some embodiments, the collection vessel and/or milk may be stored 220.

Optionally the collection vessel is sealed and/or labeled for storage. For example, a collection vessel may be sealed by removable cap (for example a screw on cap) and/or a reversible seal (for example a plastic zipper). Alternatively or additionally the collection vessel may include an irreversible seal.

In some embodiments, some components of the extraction system may be disposed of 224 after a single and/or multiple uses. For example the collection receptacle and/or the diaphragm may be disposed of 224a after a single use. Alternatively or additionally the collection vessel may be used to store milk and later disposed. For example, when a collection vessel is removed 214 from the collection receptacle, the fastener may be permanently disabled. When extraction is finished 212 the receptacle and/or fastener may be disposed of 224a. The collection vessel is then optionally used for storing the extracted milk and/or disposed of 224c when the milk is consumed.

### 3) States of a Milk Extraction System

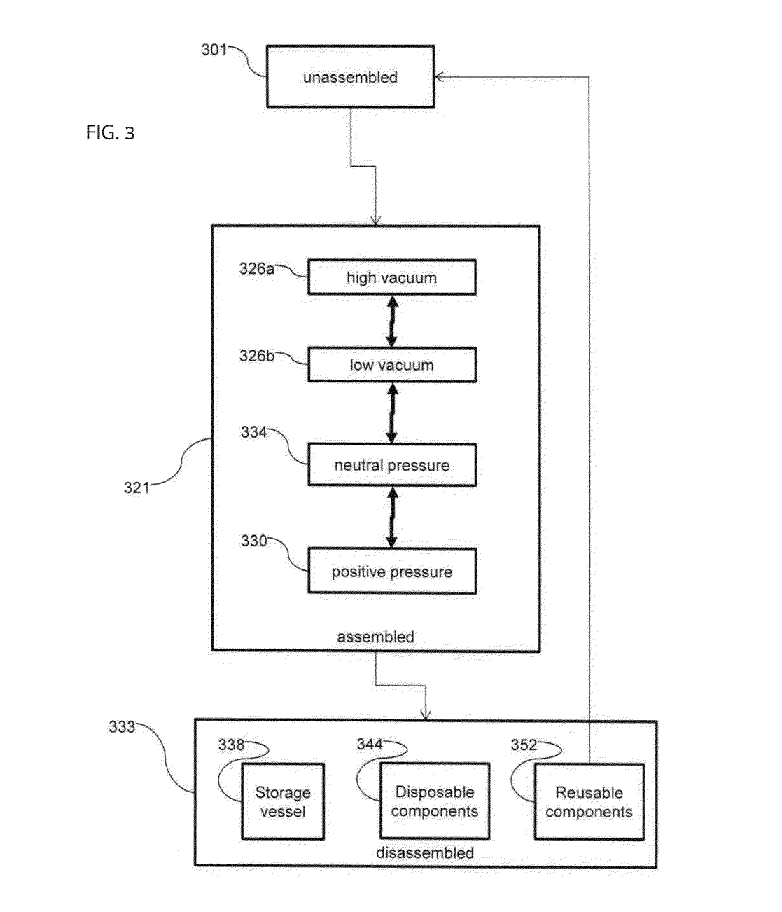

FIG. 3 is a state diagram illustrating various states of a milk extraction system in accordance with some embodiments of the present invention. Optionally, some components of the system are single use and/or disposable. For example, some components may be irreversibly assembled before use. For example some components may be irreversibly disassembled after use. Optionally disassembled components differ in number and/or form from the components prior to assembly. For example, a plastic bag collection vessel may be supplied including a storage portion and/or connecting portion for connection to an extraction receptacle and/or a diaphragm portion and/or a conduit portion. After use, the storage portion may be disconnected from the other portions (for example by cutting or ripping away the other portions). Optionally some parts are reusable. For example, a reusable part may be reversibly and or partially reversibly assembled and disassembled to the single use and/or disposable parts. For example a reusable vacuum chamber may be connected to an extraction receptacle. In some embodiments, the connection may be fully reversible (the collection chamber can be attached and/or detached to the extraction receptacle without significant irreversible change in either) and/or partially reversible (attaching and/or detaching the collection chamber to the extraction receptacle does not require a significant irreversible change in the vacuum chamber, but may be associated with a significant irreversible change in the extraction receptacle).

In some embodiments, a breast milk extraction system may have an unassembled 301 state. Optionally, in the unassembled 301 state, reusable parts may be separate from single use parts. Single use parts may include, for example, a collection vessel and/or an extraction receptacle and/or a milk conduit. Disposable parts are optionally supplied to a user as preassembled sub-system and/or as a disassembled kit and/or as separate disassembled parts. The user may then assembly parts that need assembly and/or attach reusable parts (for example a vacuum chamber) to the single use assembly to place the system into an assembled 321 state. The system may be mounted to a breast in the fully assembled 321 state and/or parts of the system may be mounted to a breast in a partially assembled state and/or in the unassembled 301 state. Optionally, assembly may be completed while the system is mounted to the breast.

Some embodiments may include reversible connections (wherein after disconnection all of the parts return to their preattached state) and/or semi reversible connections (wherein after disconnection some of the parts return to their preattached state and/or some parts are permanently changed) and/or irreversible connections (where all of the parts are permanently changed). For example a vacuum chamber may be connected to an extraction receptacle with a fully reversible clamp and/or friction fitting and/or screw thread and/or a collection vessel (for example a baby bottle) may be connected to an extraction receptacle by a screw thread. Alternatively or additionally a vacuum chamber may be connected to an extraction receptacle with a partially reversible adhesive (for example the adhesive may be mounted on the single use receptacle the adhesive may reversibly stick to a surface of the vacuum chamber such that on removal, the vacuum chamber can be reused, but the adhesive of may lose its tackiness).

Alternatively or additionally, a collection vessel may be irreversibly connected to a receptacle (for example a collection bag may be connected to the receptacle by a permanent adhesive and/or a heat seal, the bag may be removed by tearing and/or two parts may be connected by a irreversible snap and/or interference element).

In some embodiments, the assembled system may have various pressure states.

Optionally, while the system is mounted to a breast various pressure states are used to extract milk. For example, the system may have one or more vacuum 326a 326b states. Optionally, the system has a neutral 334 pressure state. Optionally, the system has one or more positive 330 pressure states. For example in the high vacuum 326a state negative pressures may range for example between 50 to 120 mmHg and/or 120 to 250 mmHg. During the high vacuum 326a and or low vacuum 326b state milk is optionally drawn out of the breast. In a neutral 334 and/or low vacuum 326b state negative pressure may range between 50 mmHg to 20 mmHg and/or between 20 to 0 mmHg. In a positive 330 pressure state the pressure may range for example between 0 to 50 mmHg positive and/or 50-120 mmHg and/or 120-250 mmHg. In the low vacuum 326b, neutral 334 and/or positive 330 pressure state, milk optionally flows to a collection vessel. For example flow may be driven by pressure gradients and/or by gravity. Optionally one or more valves control the direction and/or timing of flow. In some embodiments, the system may cycle between a high vacuum 326a and a low vacuum 326b state. In some embodiments the system may cycle between a high vacuum 326a and/or a low vacuum 326b and/or a neutral 334 state. In some embodiments the system may cycle between a high vacuum 326a and/or a low vacuum 326b and/or a neutral 334 and/or a positive 330 pressure state. The rate of cycling between pressure states may range for example between 90 to 60 cycles per minute and/or 60 to 30 cycles per minute and/or 30 to 10 cycles per minute. The cycle may optionally change over time. For example, in the beginning of extraction let down may be encouraged. Optionally in the beginning of extraction very slow cycles and/or very fast cycles and/or relatively mild negative pressures are applied. Optionally, later on during extraction, cycles are faster and/or a higher magnitude negative pressure is applied. At the end of extraction hind milk may be extracted. Optionally at the end of extraction slower cycles are used.

In some embodiments, a milk extraction system may have a disassembled 333 state. Optionally, after use, the system is disassembled into the disassembled 333 state. For example, reusable components 352 may be separated from single use components 344. Optionally, some or all of the reusable components are cleaned and/or sterilized and/or reused. In some embodiments, some reusable components may be protected from contact with the milk and/or may be reused without cleaning and/or sterilizing. For example a vacuum chamber may be separated from the milk by a diaphragm. The vacuum chamber may, in some embodiments be reused without cleaning. For example reusable components may include a vacuum chamber and/or a pump. Optionally, some single us components are disposed of after use. For example, disposable components may be thrown into the garbage. For example single use components may include a collection receptacle and/or a collection bag. Optionally in the disassembled 333 state, a collection vessel 338 may be separated from other components. In some embodiments, a collection vessel 338 may include disposable components. Alternatively or additionally a collection vessel 338 may include reusable components (for example a baby bottle and/or a nipple). For example, milk may be stored in collection vessel 338. After the milk is used, the collection vessel may be disposed of (for example by throwing it into the garbage). Alternatively or additionally, in some embodiments, after the milk is used, the collection vessel may be cleaned and/or reused.

### 4) Components of a Milk Extraction System

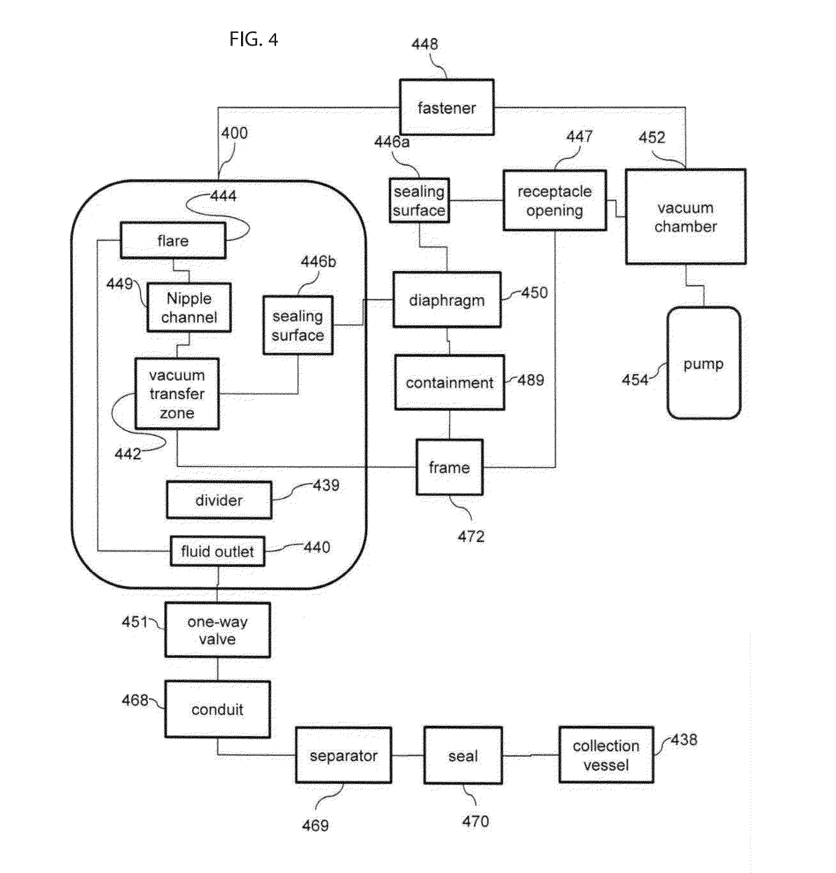

FIG. 4 is a block diagram illustrating components of a milk extraction system according to some embodiments of the current invention. The block diagram illustrates some optional connections between components. In some embodiments a diaphragm and/or conduit and/or one-way valve and/or collection vessel may be formed of polypropylene and/or polyethylene for example of thickness ranging between 80-120 micron. Optionally some or all of the diaphragm and/or conduit and/or one-way valve and/or collection vessel are formed of a single integral piece of material. Optionally the diaphragm and/or conduit and/or one-way valve and/or collection vessel are single use components. Alternatively or additionally some or all of the diaphragm and/or conduit and/or one-way valve and/or collection vessel are reusable and/or made of other materials and thicknesses. Optionally, the system may include a frame and/or a containment. For example a frame and/or a containment may limit movement of a diaphragm. For example, the frame and/or containment may protect the diaphragm from damage due to over-extension and/or the frame and/or a containment may protect the system from movement of a too large volume of air (that may for example cause damage to a nipple by sucking it too far into a tube etc.). In some embodiments, the system may include a fastener, for example connecting a vacuum chamber to an extraction receptacle. For example the fastener may rigidly connect a multiuse vacuum chamber to a single use extraction receptacle. Optionally the fastener may include an independent component and/or a portion of the extraction receptacle and/or a portion of the vacuum chamber. For example, the fastener may include reusable parts on the vacuum chamber connecting to single use parts on the extraction receptacle.

In some embodiments, a receptacle opening 447 in a vacuum chamber 452 may be in pressure communication with a vacuum transfer zone 442 in an extraction receptacle 400. Optionally, zone 442 will be partially and/or completely surrounded by a sealing surface 446b and/or opening 447 will be partially and/or completely surrounded by a sealing surface 446a. For example, surfaces 446a, b may be configured to facilitate an air tight connection between extraction receptacle 400 and a vacuum chamber 452. In some embodiments, a diaphragm 450 may prevent mass transport between vacuum chamber 452 and extraction receptacle 400. Optionally, diaphragm 450 may transfer a pressure signal between the vacuum chamber 452 and extraction receptacle 400.

In some embodiments a milk extraction system may include a vacuum chamber 452. Optionally the vacuum chamber 452 has rigid walls and/or a fixed shape. In some embodiments a pump 454 is operationally connected to vacuum chamber 452.

Optionally the pump 454 evacuates fluid (for example air) from chamber optionally creating a vacuum in chamber 452. Alternatively or additionally pump 454 pumps fluid to vacuum chamber 452 optionally creating a positive pressure in chamber 452. Optionally, the vacuum chamber may have a fixed internal volume, for example ranging between 2 and 12 cm³ and/or between 12 to 30 cm² and/or between 30 to 100 cm². In some embodiments the vacuum chamber may be molded of a single piece of plastic. Optionally the vacuum chamber includes receptacle opening 447.

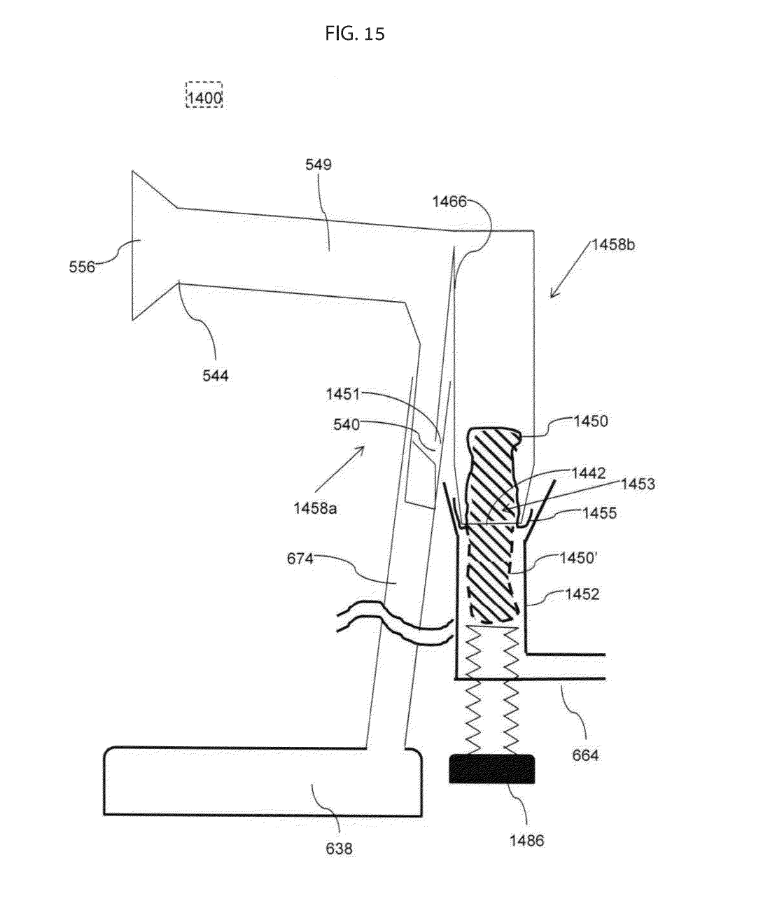

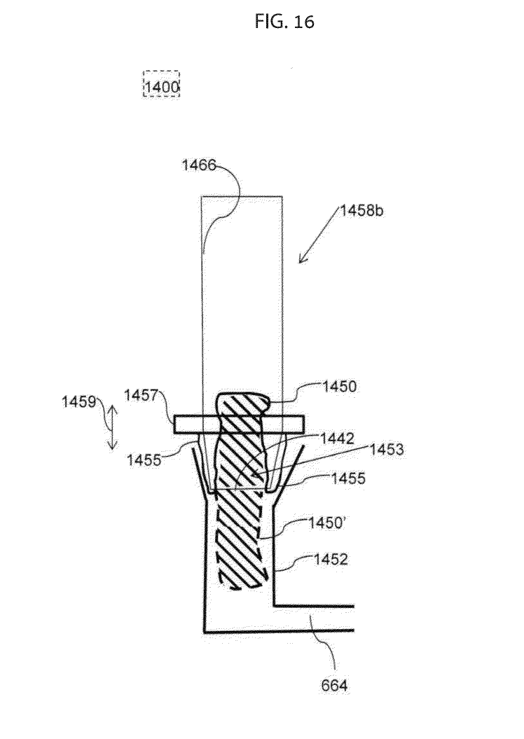

In some embodiments a fastener 448 may attach vacuum chamber 452 to extraction receptacle 400. For example, a fastener may include a clamp and/or an adhesive and/or a snap and/or a hook and loop fastener and/or a screw and/or a cam lock and/or a magnet and/or a clip and/or a pin and/or inner and outer friction connected cones (for example as illustrated in FIGS. 15 and 16) etc. Optionally, fastener 448 may include reusable and/or single use parts. For example, a reusable clamp and/or a single use snap may lock a single use lip on a receptacle to a multiple use lip on a vacuum chamber.

In some embodiments a diaphragm 450 may separate between extraction receptacle 400 and vacuum transfer zone 442. Optionally, diaphragm 450 prevents transport of fluids (for example milk or air) between receptacle opening 447 of vacuum chamber 452 and vacuum transfer zone 442 of receptacle 400. Optionally, diaphragm transmits pressure changes between receptacle opening 447 of vacuum chamber 452 and vacuum transfer zone 442 of receptacle 400. For example, diaphragm 450 may include a flexible membrane and/or an elastic membrane and/or a piston. Diaphragm 450 is optionally a single use part. For example, a single use diaphragm may be a separate part and/or part of a single use receptacle 400 and/or part of a single use collection vessel.

Alternatively or additionally, diaphragm 450 may be reusable.

In some embodiments, a breast milk extraction system may include one or more sealing surface 446a, b. For example, extraction receptacle 400 may include a sealing surface 446b, for example including a smooth lip. Alternatively or additionally, vacuum chamber 452 may include a sealing surface 446a, for example including a sealing-ring (or example an o-ring). Sealing surfaces 446a, b optionally surround transfer zone 442 and/or opening 447 respectively. For example, when the breast milk extraction system is assembled, sealing surfaces 446a, b may seal together opening 447 and/or transfer zone 442. Sealing surfaces 446a, b, may prevent fluid leakage between components of the milk extraction system, for example between vacuum chamber 452 and extraction receptacle 400. In some embodiments, diaphragm 450 and/or frame 472 may be sealed against and/or between one or both of sealing surfaces 446a, b. For example sealing surface 446a may be on an outside surface of vacuum chamber 452. Alternatively or additionally, a diaphragm may be located inside of extraction receptacle 400. For example, the diaphragm may be adhered to the inside wall of extraction receptacle 400.

In some embodiments, a breast milk extraction system may include a containment 489. Optionally, containment 489 may limit movement of a diaphragm and/or membrane. For example, frame 472 and/or containment 489 may be interconnected and/or may be attached to and/or be a part of vacuum chamber 452 and/or extraction receptacle 400. Optionally, containment 489 limits extension of diaphragm 450 in one or more directions. For example limiting extension of the diaphragm may limit the volume shifted between vacuum chamber 452 and receptacle 400. For example, containment 489 may prevent extension of diaphragm too far decreasing the volume associated with receptacle 400. For example, limiting reduction of volume of receptacle 400 may include limiting extension of diaphragm 450 into receptacle 400. Limiting movement of diaphragm into receptacle 400 may limit positive pressure fluctuations inside of receptacle 400. For example containment 489 may prevent diaphragm 450 from decreasing a volume associated with receptacle 400 by more than a limit ranging between 5 to 10 cm³ and/or ranging between 10 to 20 cm³.

Alternatively or additionally containment 489 may limit negative pressure fluctuations inside of extraction receptacle 400. For example, containment 489 may prevent movement of diaphragm too far into vacuum chamber 452 through opening 447. For example containment 489 may prevent diaphragm 450 from decreasing a volume associated with vacuum chamber 452 by more than a limited ranging between 10 to 15 cm³ and/or between 15 to 20 cm³ and or between 20 to 30 cm³. Optionally, a containment 489 may be adjustable by a user (for example to achieve extraction that is comfortable and/or effective and/or does not pinch a nipple of the user). Optionally containment 489 may be located between and/or around opening 447 and/or transfer zone 442. Alternatively or additionally, movement of diaphragm 450 may be limited to a slack of the diaphragm across a pressure transfer zone (for example zone 442). For example, diaphragm 450 may include a membrane sealed over pressure transfer zone 442. The membrane may have limited elasticity and/or not significantly stretch at pressure differentials less than 300 mmHg across the membrane. For example, then movement of diaphragm 450 and/or the volume of pressure oscillations may be limited to the slack in the membrane and/or twice the slack of the membrane.

In some embodiments, a breast milk extraction system may include a flare 444 for a breast. Optionally, flare 444 may be single use. For example flare 444 may be part of single use extraction receptacle 400. Optionally, the flare 444 and/or receptacle 400 is made of a disposable material, for example plastic, abs, and/or polypropylene.

Optionally, the material of flare 444 and/or receptacle 400 is thin, for example the wall thickness may range between 0.5 to 0.8 mm and/or between 0.8 to 1.0 mm and/or from 1.0 mm to 1.5 mm. In some, embodiments, the breast flare may have an opening for mounting on the breast. For example a diameter of the opening may range between 3 to 4.5 cm and/or 4.5 to 5.5 cm and/or from 5.5 to 7 cm. For example the smaller sized opening (for example less than 5.0 cm) may be configured to fit through a hole in a retainer to be held to the breast.

In some embodiments, an extraction receptacle may include a nipple channel 449. For example, nipple channel 449 may be thin. Optionally during a negative pressure oscillation at pressure transfer zone 442 a nipple is sucked into nipple channel 449 and/or squeezed. In some embodiments, the vacuum and/or the squeezing may extract milk from the breast. Optionally, the length and/or width of nipple channel 449 may be suited to the length that the nipple will be drawn out during milk extraction. For example the nipple channel may have a volume larger than the largest volume of a negative pressure fluctuation. The nipple channel is optionally long enough and/or has a large enough volume to house the nipple for the largest vacuum oscillation. For example a nipple channel may have a diameter ranging between 1.5 and 3.5 cm. The length of a nipple channel 449 may range for example between 5 to 15 mm and/or between 15 to 30 mm and/or between 30 to 50 mm and/or larger. The width (for example the diameter) of nipple channel 449 may range for example between 10 to 20 mm and/or between 20 to 30 mm. For a larger volume vacuum fluctuation nipple channel may be longer (for example for vacuum fluctuations of volume 20 ml or more nipple channel 449 may have a length of 4 cm or more or less. When vacuum fluctuations are limited to between 10 to 20 ml and/or less than 10 ml, nipple channel may have a length ranging between 0.5 and 2 cm.

In some embodiments, an extraction receptacle 400 may include a fluid outlet 440. For example, milk extracted into extraction receptacle 400 may flow through fluid outlet 440 to a collection and/or storage vessel 438. In some embodiments, a divider may separate a milk flow pathway from a vacuum pathway. Optionally, fluid outlet 440 may include a one-way valve. Optionally, one-way valve 451 allows milk flow into collection vessel 438 but impedes leakage of vacuum out of extraction receptacle 400 through fluid outlet 440. Optionally, one-way valve 451 may include single use parts and/or reusable parts. For example, a flap of collection vessel 438 may cover fluid outlet 440, when there is a vacuum inside extraction receptacle 400. For example, the flap may open when there is zero pressure or positive pressure for example by pushing the flap away from outlet 440, opening a flow path to a conduit 468 and/or to storage vessel 438. In some embodiments a flap of one way valve may include a part of collection vessel 438 and/or conduit 468.

In some embodiments, a breast milk collecting system may include a conduit 468. For example, conduit 468 may supply a flow pathway from fluid outlet 440 to collection vessel 438. Optionally, a flexible sleeve of collection vessel 438 may extend and/or surround a rear portion of extraction receptacle 400. Optionally a part of the sleeve may be included in diaphragm 450 and/or one-way valve 451. Alternatively or additionally, the sleeve may be included in conduit 468. Optionally, fluid conduit 468 is made of a flexible collapsible material (for example Polyethylene and/or nylon and/or polyester). In some embodiments, after collecting milk, the collection vessel may be separated from the extraction receptacle and/or the vacuum chamber. Separation may be by means of a separator 469, for example a tear line and/or a scissors and/or breaking a stiff part (for example of a friction fit and/or a threaded fastener). Milk is optionally stored in the collection vessel and/or fed to a baby from the collection vessel.

Alternatively or additionally, milk may be emptied from the collection vessel to a feeding vessel (for example a baby bottle). In some embodiments, the collection vessel will include a seal 470 for example a tie and/or a plastic zipper and/or an adhesive.

Optionally collection vessel 438 includes a label. For example, the labeling may include markers for milk quantity. Alternatively or additionally there may be place to write the day and/or time and/or name of a user. Alternatively or additionally there may be a heat sealer that may be also a printer for example to print date and time.

Alternatively or additionally there may be indicators (for example stickers and/or tape on a collection bag) for temperature measurement and/or milk quality. Alternatively or additionally a milk collection bag shape may be similar to the size of a bottle so that for a given milk height, the volume in the bag will be similar to the quantity of milk in a bottle filled to the same height. Optionally a collection vessel may have storage capacity of between 30 to 60 ml and/or 60 to 90 ml and/or 90 to 120 ml and/or 120 to 200 ml.

### 5) An Extraction Receptacle

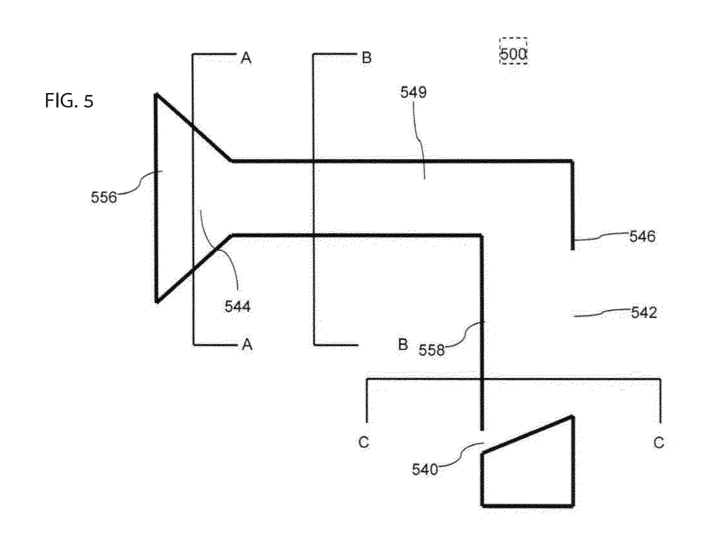

FIG. 5 is a schematic cross-sectional view of a milk extraction receptacle 500 in accordance with some embodiments of the current invention. Receptacle 500 optionally has a simple body in the form of a bent tube with multiple openings. For example, receptacle 500 may include on a first end a breast flare 544 having a breast opening 556 configured to mount to a breast. Optionally the second end of flare 544 is connected to a nipple channel 549. Optionally extraction receptacle 500 may include a flow control section 558. For example, receptacle 500 may include a vacuum transfer zone 542 and fluid outlet 540. For example vacuum transfer zone 542 may include an opening connectable to a vacuum chamber. Alternatively or additionally vacuum transfer zone 542 may include multiple openings and/or a grating. Optionally, a sealing surface 546 surrounds vacuum transfer zone 542. Optionally, flow control section 558 may include fluid outlet 540 and/or vacuum transfer zone 542. Alternatively or additionally, a vacuum transfer zone and/or a fluid outlet may be located in the breast flare. Optionally, flow control section 558 may be included in an extension of receptacle 500.

In some embodiments the cross sectional geometry of an extraction receptacle may change along its length. For example, a cross section of the breast flare near the sealing surface 546, for example at A-A, may be circular and/or elliptical. For example, across of the breast flare further from opening 556, for example along nipple channel 549 and/or at B-B, may be circular and/or elliptical with smaller dimension and/or a different orientation than at A-A. A cross section of the flow control section 558 for example near the vacuum transfer zone 542, for example at C-C, may be rectangular and/or half circular or of another cross section. Alternatively, the cross section geometry may be circular along the entire length of receptacle 500.

### 6) Operation of an Extraction Receptacle

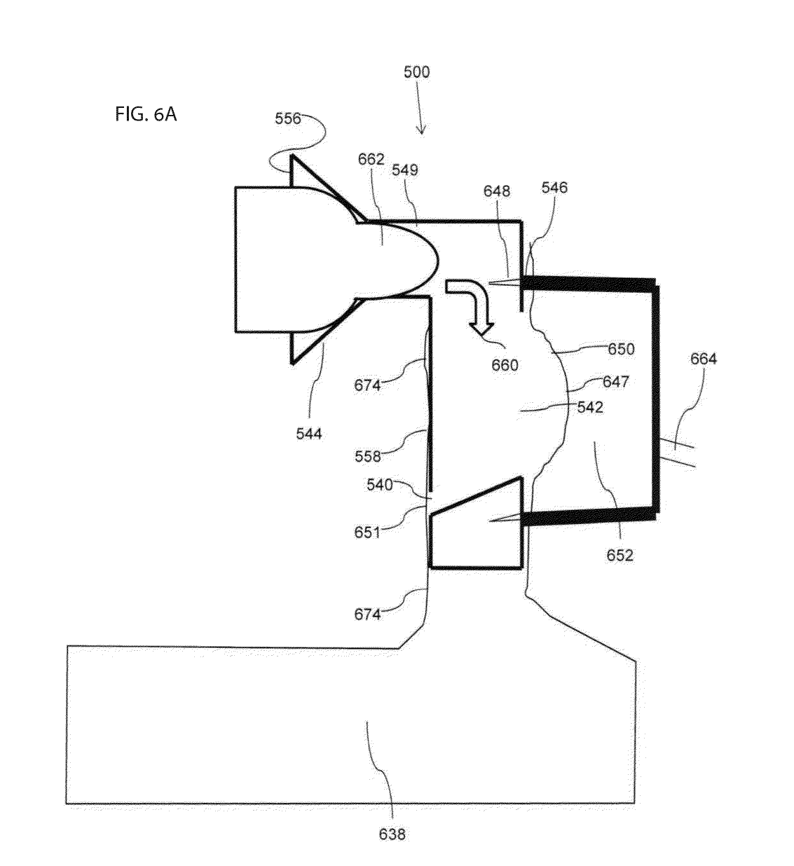

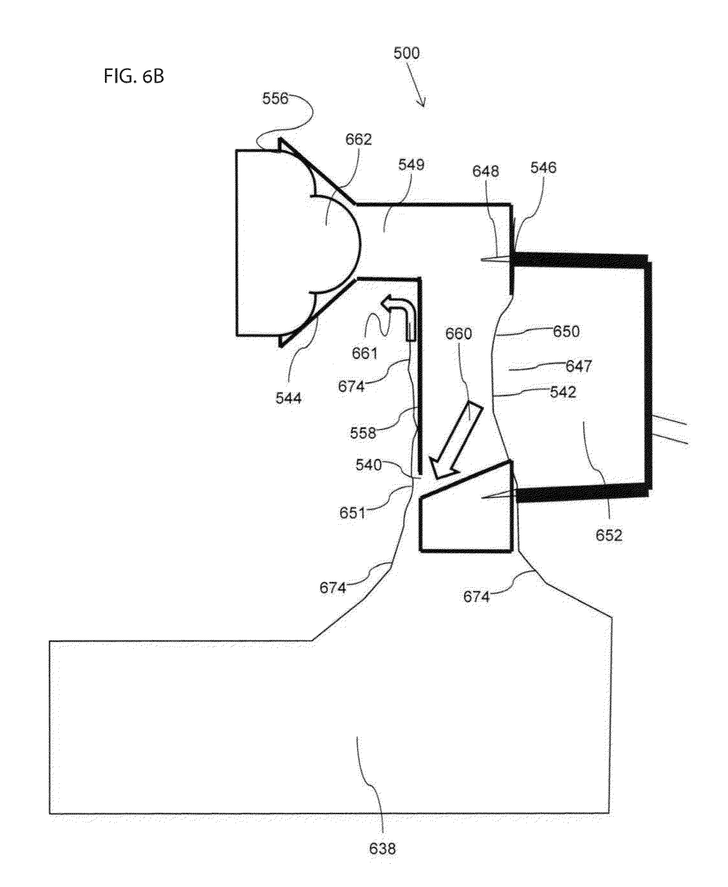

FIGS. 6A and 6B are schematic cross sectional views of a breast milk collection system in negative pressure and positive pressure states respectively in accordance with an embodiments of the current invention. Optionally, a flexible milk collection vessel 638 may include a diaphragm 650 and/or a valve flap 651. Optionally, portion of collection vessel 638 (for example a sleeve 674 extending from collection vessel 638) surrounds a portion of extraction receptacle 500. For example, sleeve 674 may include diaphragm 650 and/or valve flap 651. Optionally diaphragm prevents mass transfer across vacuum transfer zone and/or transmits pressure signals across vacuum transfer zone 542. For example, diaphragm 650 may cover vacuum transfer zone 542 of receptacle 500. For example, diaphragm 650 moves back and forth in the space dividing vacuum chamber 652 from receptacle 500.

In some embodiments, diaphragm 650 may be sandwiched between vacuum chamber 652 and receptacle 500. For example, diaphragm 650 may be spread across vacuum transfer zone 542 of receptacle 500 and/or receptacle opening 647 of vacuum chamber 652. Optionally diaphragm 650 may be sealed against sealing surface 546. Vacuum chamber 652 is optionally rigidly attached to receptacle 500 for example by a friction fit pin 648 and/or a clamp. Vacuum chamber 652 is optionally connected to a pressure and/or vacuum source (for example a pump) by a vacuum tube 664.

FIG. 6A, illustrates a system for extracting milk in a negative pressure state in accordance with some embodiments of the current invention. Optionally, receptacle 500 is mounted to a breast which blocks air flow through flare 544 and or flare opening 556.

Optionally negative pressure is applied to vacuum chamber 652 via vacuum tube 664. In the example of FIG. 6A, negative pressure may deform diaphragm towards vacuum chamber 652 enlarging the internal volume of receptacle 500. Increased internal volume may, in some embodiments, create a vacuum inside of receptacle 500 and/or transfer the vacuum from vacuum chamber 652 to receptacle 500. Optionally, the vacuum sucks flap 551 against fluid outlet 540 closing the outlet. Alternatively or additionally, a separate one-way valve may be supplied between receptacle 500 and collection vessel 638. Alternatively or additionally, there may not be a one way valve between receptacle 500 and collection vessel 638. For example collection vessel 638 may be rigid and/or not act as a pressure sink. Optionally sleeve 674 may be configured to permit one way flow. For example, sleeve 674 and/or vessel 638 may be made of flexible material such that when exposed to a vacuum sleeve 674 collapses and prevents flow between receptacle 500 and collection vessel 638.

In some embodiments, the vacuum draws a nipple into a nipple channel 549 and/or squeezes the nipple and/or draws milk out of breast 662. The milk optionally flows along a flow path 660 from flare 544 towards fluid outlet 540. Flow may be for example driven by the pressure (for example created by the vacuum) and/or flow may be by gravity. Milk is optionally separated from pressure chamber 652 by diaphragm 650.

FIG. 6B illustrates a system for extracting milk in a positive pressure state in accordance with some embodiments of the current invention. Optionally, positive pressure inside vacuum chamber 652 extends diaphragm 650 into extraction receptacle 500. Extending diaphragm 650 into receptacle 500 may increase pressure and/or release the vacuum inside receptacle 500. Reduced pressure inside receptacle 500 may allow breast 662 to retract and/or release squeezing force on the nipple. Positive and/or neutral pressure in receptacle 500 may in some embodiments allow flap 651 to move away from outlet 540 and/or open outlet 540. Pressure and/or gravity may drive milk along an outflow path 660 for example to a conduit (for example sleeve 674 and/or to collection vessel 638).

In some embodiments, when a vacuum is applied to receptacle 500, excess pressure may be released. For example, in FIG. 6B pressure may be released by air flow 661 between sleeve 674 and receptacle 500. Alternatively or additionally excess pressure may be released via a one way valve, for example on receptacle 500 and/or on collection vessel 638 and/or on vacuum chamber 652. Alternatively or additionally, there may not be a pressure release. For example pressure chamber 652 may not have a positive portion of the cycle and/or air may be allowed to enter collection vessel 638 and/or there may be a trap that allows fluid out of outlet 540 but prevents airflow into collection vessel.

### 7) Extraction Receptacle with Divider

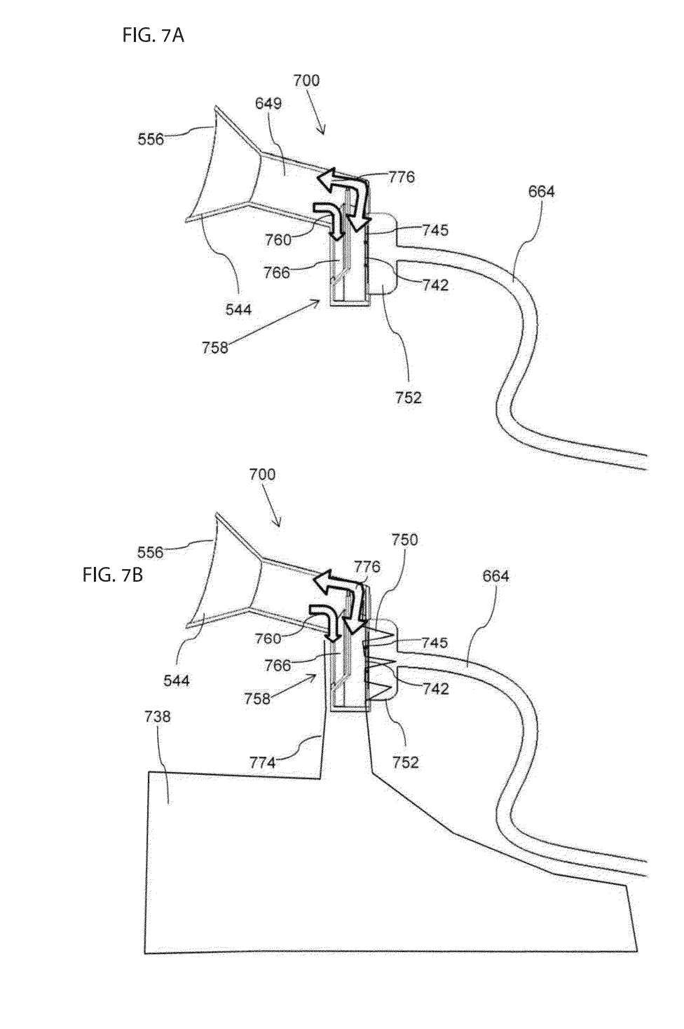

FIGS. 7A and 7B illustrate a cutaway view of an alternative embodiment of a breast milk extraction system in accordance with some embodiments of the current invention. In FIGS. 7A and 7B the outer walls of extraction receptacle 700 are illustrated cut away to show the inner structure. A flow control section 758 optionally includes a divider 766 that may serve for example to separate a fluid flow path 760 between a breast flare 544 and a fluid outlet from a pressure communication path 776 between a breast flare 544 and a transfer zone 742. Pressure chamber 752 optionally includes a grating 745 at the transfer zone 742 opening.

In some embodiments, milk flows along the bottom of flare 544 to fluid pathway 760. Fluid pathway 760 optionally leads to an outlet Pressure signals are optionally transmitted from diaphragm 750 around and/or above divider 766. A sleeve 774 of a collection vessel 738 may act as a flap for a one way valve across the outlet and/or as a pressure release and/or as a one-way conduit, for example in a manner similar to that illustrated in FIGS. 5A and 5B. Flow out the outlet may be for example by gravity flow and/or by positive pressure. Sleeve 774 optionally includes a flexible conduit. For example under negative pressures sleeve 774 may collapse, for example preventing vacuum from reaching collection vessel 738. Under positive and/or neutral pressure sleeve 774 may pass milk from receptacle 700 to collection vessel 738. The flexible conduit may optionally be longer than collection vessel 738. For example the conduit may be longer than collection vessel 738 by between 1-10 cm and/or 10 to 30 cm and/or more than 30 cm.

FIG. 7B illustrates extension a diaphragm through a vacuum transfer zone 742 according to an embodiment of the current invention. For example, vacuum transfer zone 742 may include a grating 745 and/or multiple holes. For example diaphragm 750 is shown being sucked by a vacuum in vacuum chamber 752 through small holes in grating 745. A vacuum may be transferred from vacuum chamber 752 into receptacle 700. For example, as diaphragm 750 is drawn into vacuum chamber 752, the air may be drawn behind diaphragm 750 from receptacle 700 into vacuum chamber 752.

Optionally, diaphragm 750 is partially supported by grating 745. Optionally, diaphragm 750 may be of a thinner more flexible material than diaphragm 650. Diaphragm 750 is optionally a part of sleeve 774. Alternatively or additionally, sleeve 774 may be wrapped around the flow control portion of receptacle 700 and/or sandwiched between vacuum chamber 752 and receptacle 700 for example similar to corresponding parts of FIG. 6A, B.

### 8) Fixing a Vacuum Chamber to a Extraction Receptacle

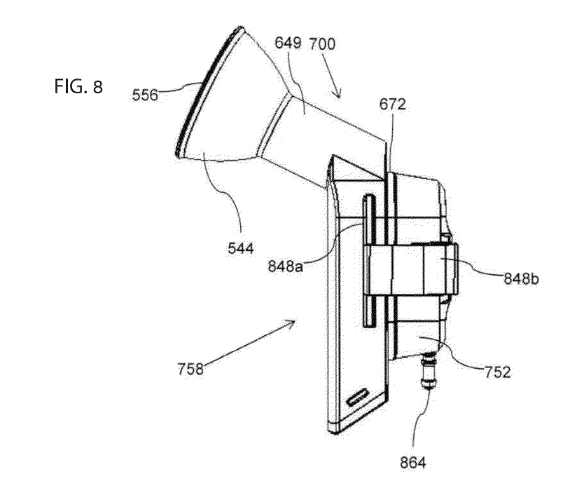

FIG. 8 illustrates an external view of extraction receptacle 700 connected to vacuum chamber 752. Optionally, vacuum chamber 752 is held fixed and/or substantially immobile with respect to receptacle 700 by a fastener 848a, b. For example fastener 848a, b may include a fastener 848b connected to a fastener 848a on receptacle 700. For example, fastener 848a includes a groove (for example as illustrated more clearly in FIG. 9). Optionally, vacuum chamber 752 includes a tubing connector 864, for example, for connection to a pump.

### 9) External View of Extraction Receptacle

FIG. 9 is a perspective view of breast milk receptacle 700 in accordance with some embodiments of the current invention. For example, a vacuum transfer zone 942 of receptacle 700 may include a rectangular hole surrounded by a sealing surface 946. An optional frame 972 is shown connected to sealing surface 946. Frame 972 in some embodiment may connect the bag used for collecting milk, and diaphragm. The frame in some embodiments may snap to receptacle 700 and hold the bag tight, for example so no other mean of connection is needed.

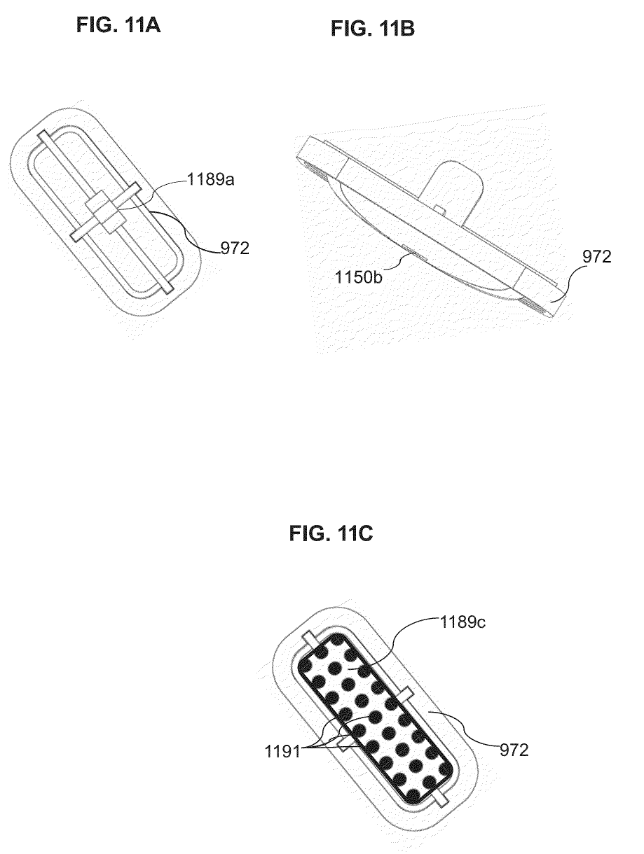

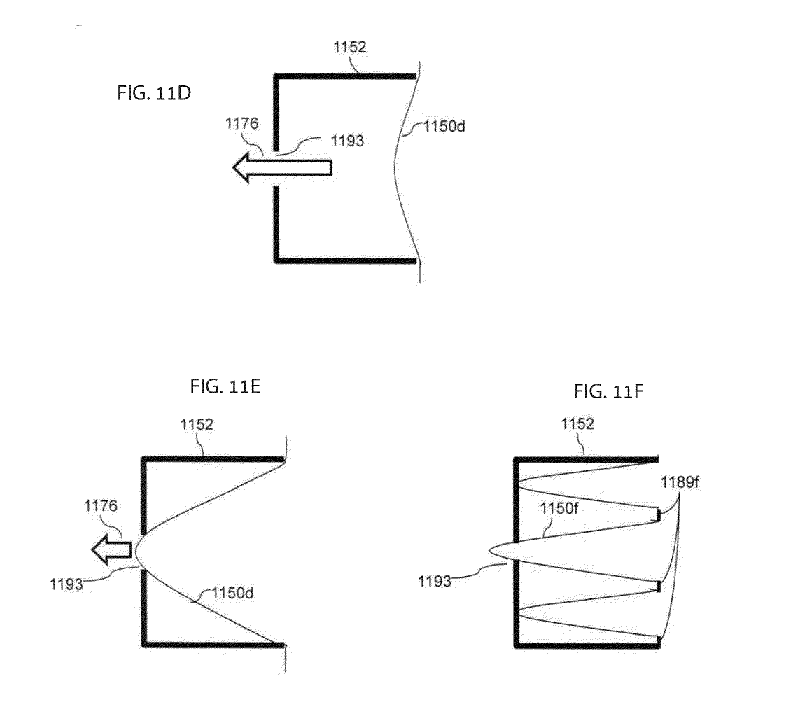

In some embodiments, receptacle 700 may be a single use part and/or may be made of inexpensive materials (for example polyethylene). Optionally, frame 972 may be a multi-use part and/or a single use part. Alternatively or additionally, vacuum transfer zone 942 may be covered by a grating and/or have a different shape and/or include a containment to restrict movement of a diaphragm (for example as illustrated in FIGS. 11A-F).

### 10) Connection Between Receptacle and Collection Vessel

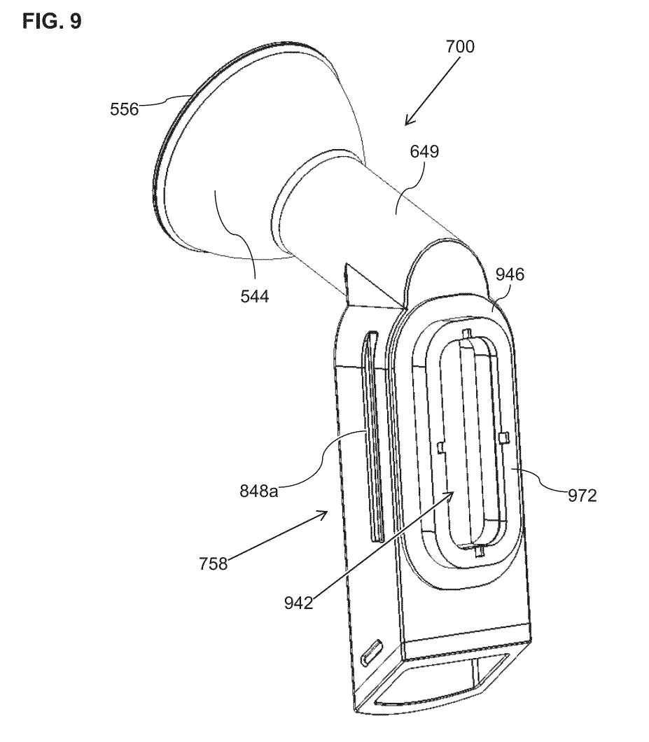

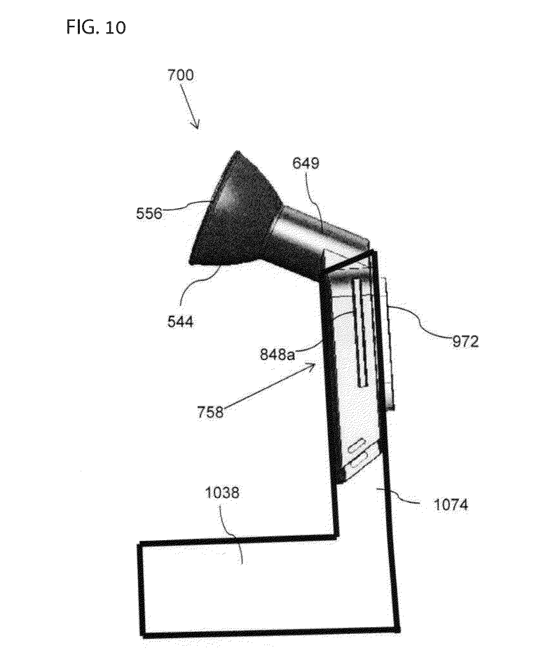

FIG. 10 is a perspective illustration of breast milk receptacle 700 attached to an exemplary flexible milk collection and storage vessel 1038 in accordance with an embodiment of the current invention. A sleeve 1074 of vessel 1038 is fit around an extension of receptacle 700. For example, in FIG. 10, sleeve 1074 is shown fit around flow control section 758. Optionally Pressure and/or friction between sleeve 1074, vessel 1038 and/or frame 972 may hold sleeve 1074 connected to receptacle 700.

In some embodiments, frame 972 may include a polyethylene single-use part that is permanently attached to sleeve 1074. Alternatively or additionally frame 972 may be a reusable part. For example, frame 972 may include an elastomer O-ring. The O-ring is optionally placed on the outside of a diaphragm. For example, a diaphragm may be sandwiched in place between one side of the O-ring and sealing surface 946. The other side of the O-ring may be sealed to a vacuum source (for example as illustrated in FIG. 8). Alternatively or additional, the O-ring may be attached to the vacuum source and/or the vacuum source may be attached to receptacle 700 and/or the O-ring may be sandwiched in between receptacle 700 and frame 972. For example, the diaphragm may include a portion of collections vessel 1038 and/or sleeve 1074. Alternatively or additionally, the diaphragm may include a discrete part separate from collection vessel 1038, and/or receptacle 700 and/or frame 972. For example, a diaphragm may include an elastic material and/or may include an inelastic material. For example, diaphragm may be mounted over vacuum transfer zone 942 with a slack that allows movement of the diaphragm.

### 11) Limiting a Vacuum Signal Volume

FIGS. 11A-F illustrate some means to limit movement of a diaphragm in accordance with some embodiments of the current invention. For example, movement of a diaphragm may be limited by a containment and/or the diaphragm may be attached across the opening with a slack allowing a limited movement of the diaphragm and/or a grating may limit movement of a diaphragm across vacuum transfer zone 942.

Alternatively or additionally, after a predetermined displacement, a diaphragm may partially and/or completely obstruct a vacuum aperture, impeding and/or preventing movement of further volume between the receptacle and the vacuum source.

FIG. 11A illustrates frame 972 connected to a containment 1189a in accordance with some embodiments of the current invention. Containment 1198a may prevent a diaphragm from being displaced too far in the direction of a vacuum source. Limiting movement of the diaphragm may limit the volume of a pressure signal transferred between a vacuum chamber and a breast extraction receptacle.

FIG. 11B illustrates frame 972 attached to a diaphragm 1150b in accordance with an embodiment of the current invention. Optionally, diaphragm 1150b may be attached with a limited freedom of movement. For example, diaphragm 1150b may be adhered to frame 972 in such a way that when a pressure differential is applied to diaphragm 1150b a predetermined volume is displaced, for example due to elasticity and/or slack. The volume of displacement may have little sensitivity to the magnitude of the pressure differential. For example, diaphragm 1150b may be substantially inelastic for pressure differentials less than 300 mmHg. A pressure differential ranging between 30 to 300 mmHg may move diaphragm to the same degree, for example as much as is allowed by a fixed slack. For example, diaphragm 1150b may be limited to allow volume a fixed volume associated with pressure transfer. The fixed volume may range for example between 5 and 15 ml and/or between 15 and 40 ml and/or greater than 40 ml.

Alternatively or additionally, movement of diaphragm 1150b may depend on a magnitude of a pressure differential. For example, diaphragm 1150b may move according to fixed slack ranging between 0 to 50 ml for any pressure differential and diaphragm 1150b may move an additional amount proportional to the pressure differential due to elastic stretching. For example, the movement due to stretching may add between 0 to 50 ml of movement at a pressure differential of 300 mmHg and proportionally less for lower pressure differentials.

FIG. 11C illustrates frame 972 attached to a grating 1189c with a plurality of small holes 1191. The surface area of the transfer zone may be defined as the sum of the area of all of holes 1191. One or more diaphragms may be stretched across the vacuum transfer area. The diaphragms may move according to example as a result of stretching and or slack. The total movement of the diaphragm may be limited by the position of grating 1189c and/or the size, shape and/or position of holes 1191.

FIGS. 11D-F illustrate a diaphragm whose movement due to application of a vacuum is limited to a predetermined volume at which the diaphragm obstructs a vacuum aperture, impeding and/or preventing further application of the vacuum in accordance to some embodiments of the current invention.

FIGS. 11D and 11E are schematic cutaway views of a vacuum chamber 1152 illustrating a vacuum 1176 drawing a diaphragm 1150d into a vacuum chamber 1152 to obstruct a vacuum aperture 1193 in accordance with an embodiment of the current invention. For example, in FIG. 11D vacuum 1176 has drawn diaphragm 1150d slightly into chamber 1152. Optionally as vacuum 1176 draws more air out of vacuum chamber 1152 diaphragm is drawn towards a vacuum aperture 1193. When the air removed from chamber 1152 reaches the predetermined critical volume, diaphragm 1150d obstructs aperture 1193 (for example as illustrated in FIG. 11E). With aperture 1193 obstructed, application of further vacuum does not significantly move diaphragm 1150d.

FIG. 11F illustrates is a schematic cutaway view of a vacuum chamber 1152 illustrating a vacuum 1176 drawing a diaphragm 1150f into a vacuum chamber 1152 to obstruct a vacuum aperture 1193 in accordance with an embodiment of the current invention. For example, in FIG. 11F, diaphragm 1150f is drawn through three holes of a grating 1189f. When the displaced volume reaches a predetermined critical level, diaphragm 1150f optionally blocks aperture 1193. Optionally, further application of vacuum will not cause further significant movement of diaphragm 1150f.

### 12) Alternate Collection Vessel

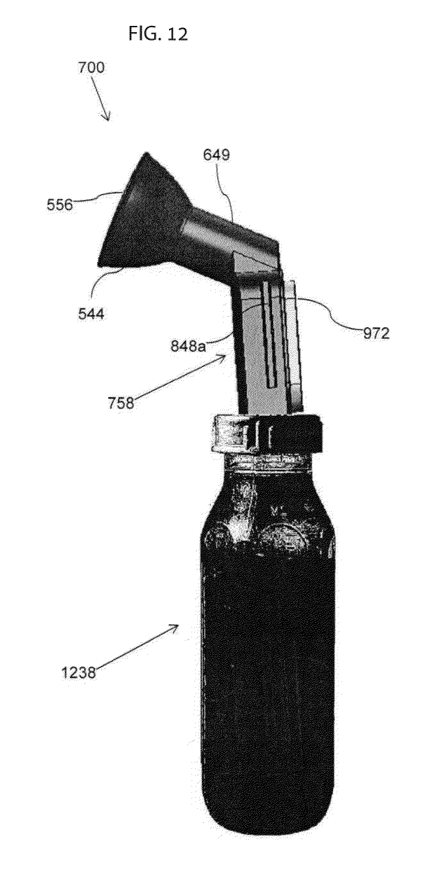

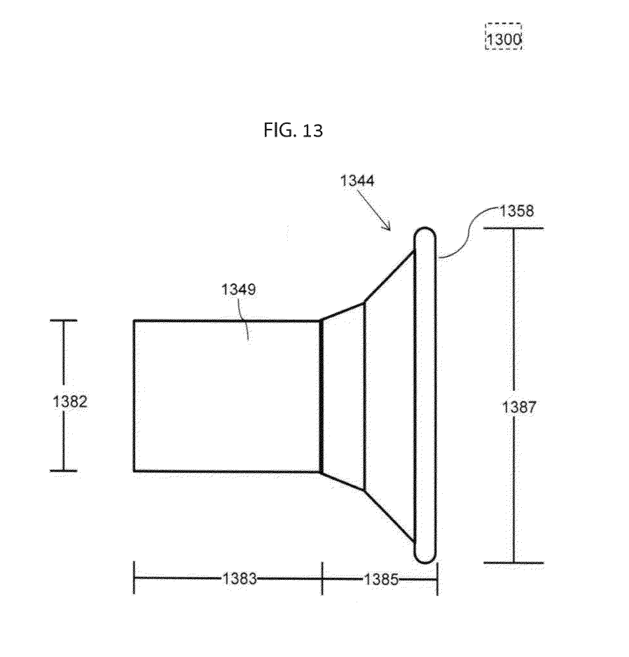

FIG. 12 illustrates a milk extraction receptacle 700 connected to a rigid milk collection and storage vessel 1238 in accordance with some embodiments of the current invention. For example, in some embodiments of the current invention, milk may be extracted directly into a baby bottle.

### 13) Dimensions of an Extraction Receptacle

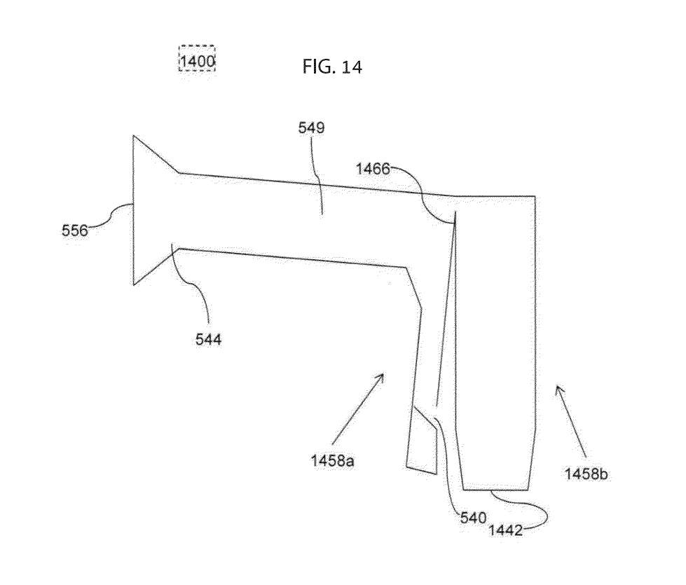

FIG. 13 is a schematic drawing illustrated dimensions of a receptacle of a milk extraction system in accordance with some embodiments of the present invention. Receptacle 1300 may be used with various embodiments of the current invention for example in the embodiments described herein above and below. In some embodiments, the width 1387 (for example the diameter) of the breast opening 1358 of receptacle 1300 may range between 35 to 55 mm and/or between 55 to 65 mm and/or between 65 to 80 mm. In some embodiments, single use receptacle and/or a receptacle may be held to a breast by a retainer may have a smaller breast opening than standard breast receptacles (for example the smaller opening may be easier to fit through an opening of the retainer and/or because the retainer holds the receptacle to the breast, the receptacle may be more stable with a smaller opening). The length 1385 of flare 1344 (which is for example a tapered portion of receptacle 1300 for mounting on the breast) may range, for example between 10 to 20 mm and/or between 20 to 40 mm. The length 1383 of a nipple channel 1349 of receptacle 1300 may range for example between 5 to 15 mm and/or between 15 to 30 mm and/or between 30 to 50 mm and/or larger. For a receptacle used with a breast massager and/or a volume limiting diaphragm, nipple channel 1349 may be shorter than a standard collection receptacle. For example, massaging and/or limiting vacuum signal volume may facilitate extraction with reduced vacuum. The nipple will optionally be pulled less deeply into nipple channel 1349. The width 1382 (for example the diameter and or the longest lateral dimension) of nipple channel 1349 may range for example between 10 to 20 mm and/or between 20 to 30 mm.

### 14) Alternate Extraction Receptacle

FIG. 14 is a schematic illustration of a milk extraction receptacle 1400 in accordance with an embodiment of the current invention. Optionally receptacle 1400 includes a divider 1466 separating between two sections 1458a, b. Optionally, the first section 1458a includes an extension for connecting to the milk collection vessel and/or a flow path for extracted milk. Optionally, the second section 1458b includes a vacuum transfer zone 1442.

### 15) Non-Elastic Diaphragm

FIG. 15 is a schematic illustration of the milk extraction receptacle 1400 connected to a pressure source (for example a vacuum chamber 1452) and/or a collection vessel (for example collection chamber and/or storage vessel 638) according to some embodiments of the current invention.

In some embodiments a diaphragm 1450 may transmit pressure signals across vacuum transfer zone 1442. For example, a pressure chamber 1452 may be attached to a vacuum transfer zone 1442 of receptacle 1400. Attachment may include sliding an end of vacuum chamber over a vacuum transfer end of a section 1458b of the receptacle. Diaphragm 1450 may be held in place, for example, by sandwiching an edge of the diaphragm in the friction fitting. Diaphragm 1450 optionally includes a seal 1455. For example when sandwiched between vacuum chamber 1452 and section 1458b of receptacle 700, seal 1455 may seal the connection (for example preventing a pressure leak) and/or increase friction to prevent a friction fit between vacuum chamber 1452 and section 1458b from coming loose. For example, the edges of diaphragm 1450 may be sandwiched between a pressure chamber 1452 and section 1458b of breast receptacle 1400.

In some embodiments, diaphragm 1450 may include a flexible but substantially inelastic membrane (for example the volume change due to flexing of the membrane is much greater [for example more than ten times] the volume change due to stretching of the membrane). For example, when pressure in vacuum chamber 1452 changes from a positive gauge pressure to a negative gauge pressure, diaphragm 1450 may move to the position of diaphragm 1450′. The volume 1453 between the two positions of diaphragm 1450/1450′ (indicated by diagonal hatching) is the volume associated with the pressure signal and/or twice the slack of diaphragm 1450. Alternatively or additionally, movement of diaphragm 1450 may be limited by a confinement and/or a grating and/or a obstructing of a pressure communication pathway. Alternatively or additionally, the volume transmitted may vary according to the magnitude of the pressure signal. For example the diaphragm may have significant elasticity.

In some embodiments, receptacle 1400 may be connected to a collection vessel. For example, milk may flow into collection vessel 638 through a fluid outlet 540. For example a sleeve 674 of collection vessel 638 may be mounted onto an extension (for example section 1458a) of receptacle 1400. Sleeve 674 optionally includes a flexible conduit. For example under negative pressures, sleeve 674 may collapse, for example preventing vacuum from reaching collection vessel 638. Under positive and/or neutral pressure sleeve 674 may pass milk from receptacle 1400 to collection vessel 638. The flexible conduit may optionally be longer than collection vessel 638. For example the conduit may be longer than collection vessel 638 by between 1-10 cm and/or 10 to 30 cm and/or more than 30 cm. When there is a positive gauge pressure inside receptacle 1400, a flap 1451 of sleeve 674 may be pushed away from outlet 540 allowing milk to flow into sleeve 674 and/or collection vessel 638. When there is a negative gauge pressure inside receptacle 1400, sleeve 674 may be pulled against outlet 540 and/or collapse prevent suction from reaching the storage/collection area of vessel 638. Receptacle 1400 optionally includes a pressure and/or volume regulator 1486. For example regulator 1486 may screw into and/or out of vacuum chamber 1452. Optionally, screwing regulator 1486 inward may partially block vacuum tube 667 reducing the amplitude of vacuum fluctuations in vacuum chamber 1452. In some embodiments regulator 1486 may not interfere with vacuum tubing 667 and/or a vacuum signal.

Alternatively or additionally, as regulator 1486 is screwed in, it may limit movement of diaphragm 1450 and/or limit the volume of pressure fluctuations transferred between vacuum chamber 1452 and receptacle 700.

### 16) Fluctuation Volume Limiter

FIG. 16 is a schematic illustration of the milk extraction receptacle 1400 connected to a pressure source including an alternative pressure fluctuation volume limiter 1457 in accordance with some embodiments of the current invention. In the embodiment of FIG. 16, the seal 1455 portion of diaphragm 1450 is extended to attach to a handle 1457. Optionally pulling 1459 up or down on handle 1459 increases and/or decreases the volume of pressure fluctuations. For example, when handle 1459 is pulled up, the ends of diaphragm 1430 are pulled out of receptacle 1400 and/or slack volume 1453 of diaphragm 1450 is decreased. For example, decreasing slack volume 1453 optionally decreases the volume of pressure fluctuations transferred by diaphragm 1450. For example, when handle 1459 is pushed down, the ends of diaphragm 1430 are pushed into receptacle 1400 and/or slack volume 1453 of diaphragm 1450 is increased. For example, increasing slack volume 1453 optionally increases the volume of pressure fluctuations transferred by diaphragm 1450.

It is expected that during the life of a patent maturing from this application many relevant technologies will be developed and the scope of the terms are intended to include all such new technologies a priori.

As used herein the term “about” refers to ±10%

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A receptacle for breast milk extraction using an external vacuum source comprising:
an open end for receiving a human breast; and
a vacuum transfer zone on an outer surface of the receptacle, said vacuum transfer zone having a surface area of at least 1 cm² and said vacuum transfer zone being in pressure communication with an interior of the receptacle and the external vacuum source.

2. The receptacle of claim 1, further comprising:
a deformable diaphragm blocking fluid communication between said interior of the receptacle and said external vacuum source wherein said deformable diaphragm is larger than said surface area and said deformable diaphragm is fixed across said vacuum transfer zone with a slack and wherein said slack is greater than an elasticity of said deformable membrane under a pressure differential of less than 250 mmHg across said membrane.

3. The receptacle of claim 1, further comprising:
a deformable diaphragm blocking fluid communication between said interior of the receptacle and said external vacuum source wherein said deformable diaphragm is larger than said surface area and said deformable diaphragm is fixed across said vacuum transfer zone with a slack and wherein a movement of said deformable diaphragm is substantially limited to said slack.

4. The receptacle of claim 1, further comprising:
a deformable diaphragm blocking fluid communication between said interior of the receptacle and said external vacuum source wherein said deformable diaphragm includes a portion of a collection vessel.

5. (canceled)

6. The receptacle of claim 1, further comprising:
a deformable diaphragm blocking fluid communication between said interior of the receptacle and said external vacuum source, and
a containment for restricting movement of said deformable diaphragm to limit a volume shift between said receptacle and said external vacuum source associated with a pressure fluctuation transferred between said external vacuum source and said interior of the receptacle.

7. The receptacle of claim 1, further comprising:
a deformable diaphragm blocking fluid communication between said interior of the receptacle and said external vacuum source, and a vacuum aperture along a pathway of said pressure communication and wherein said diaphragm obstructs said vacuum aperture when a volume shift between said receptacle and said external vacuum source associated with a pressure fluctuation surpasses a predetermined volume.

8. The receptacle of claim 1, further comprising:
a connection configured for sealing to a milk collection vessel during said extraction such that the only opening between said milk collection vessel and an exterior of the receptacle is said open end.

9. The receptacle of claim 1, wherein a width of said open end is less than 50 mm.

10. The receptacle of claim 1, further comprising:
a nipple chamber and wherein when a vacuum is applied to said vacuum transfer zone, a nipple of said breast is drawn into said nipple chamber and wherein a volume of said nipple chamber is less than 20 ml.

11. (canceled)

12. The receptacle of claim 10, further comprising:
a diaphragm configured for:
blocking fluid communication between said opening and said external vacuum source and
limiting a volume shift between said receptacle and said external vacuum source associated with a pressure fluctuation transferred between said external vacuum source and said interior of the receptacle, wherein said diaphragm is configured for said limiting of said volume shift to less than a said volume of said nipple chamber.

13-16. (canceled)

17. The receptacle of claim 1, further comprising:
a vacuum chamber rigidly connected to said outer surface and covering said vacuum transfer zone and wherein an inner volume, of said vacuum chamber is in pressure communication with said vacuum transfer zone and said external vacuum source; and
a coupling reversibly restraining said vacuum chamber immobile with respect to said receptacle.

18. The receptacle of claim 1, further comprising:
a vacuum chamber rigidly connected to said outer surface and covering said vacuum transfer zone and wherein an inner volume, of said vacuum chamber is in pressure communication with said vacuum transfer zone and said external vacuum source;
a fluid outlet; and
a milk collection vessel receiving a fluid flow from said fluid outlet said milk collection vessel including a flexible portion covering a said vacuum transfer zone; said flexible portion blocking fluid communication between said interior of the receptacle and said vacuum chamber.

19. The receptacle of claim 18, wherein said flexible portion covers said fluid outlet allowing flow through said fluid outlet from said receptacle to said milk collection vessel and preventing flow through said fluid outlet from said milk collection vessel to said receptacle.

20. (canceled)

21. A method of extracting milk from a human breast using an external vacuum source comprising:
mounting a receptacle to a breast, a portion of said breast received into an interior of said receptacle;
shifting a volume between said receptacle and the external vacuum source to transmit a vacuum pressure fluctuation from external vacuum source to said interior of said receptacle and the breast thereby extracting the milk;
blocking with a diaphragm fluid flow between said interior of said receptacle to the external vacuum source, and
limiting said shifted volume to be less than a predetermined value by said diaphragm.

22. (canceled)

23. The method of claim 21, wherein said diaphragm is substantially non-elastic in a vacuum range between 50 and 250 mmHg and said limiting includes restricting movement of said diaphragm to substantially to twice a volume of a slack in said diaphragm.

24. The method of claim 21, wherein said shifting is via a diaphragm said limiting includes:
obstructing a path of said vacuum signal by said diaphragm when said shifted volume reaches said predetermined value.

25-29. (canceled)

30. The method of claim 21, wherein said blocking is by diaphragm including a portion of a collection vessel.

31-42. (canceled)

43. A collection bag for a milk extraction receptacle comprising:
a milk collection vessel; and
a flexible conduit attachable to the extraction receptacle, said flexible conduit collapsing when a vacuum is applied from said extraction receptacle to prevent transmission of said vacuum to said collection vessel.

44. The collection bag of claim 43, wherein said flexible conduit has a wall thickness between 80 to 120 micron.

45. The collection bag of claim 43, wherein said flexible conduit is longer than said collection vessel.

46-50. (canceled)
